(19) United States
(12) Patent Application Publication
(10) Pub. No.: US 2013/0230511 A1
(43) Pub. Date: Sep. 5, 2013

(54) BIOMARKERS FOR RESPONSE TO TYROSINE KINASE PATHWAY INHIBITORS IN CANCER

(71) Applicant: Board of Regents, The University of Texas System, (US)

(72) Inventors: John V. HEYMACH, Pearland, TX (US); Ignacio I. WISTUBA, Houston, TX (US); Monique B. NILSSON, Sugar Land, TX (US); Lauren A. BYERS, Houston, TX (US); Ximing TANG, Houston, TX (US); Fei YANG, Houston, TX (US); Edward KIM, Houston, TX (US); Anne S. TSAO, Houston, TX (US); Scott M. LIPPMAN, Houston, TX (US); Waun Ki HONG, Houston, TX (US); Roy S. HERBST, Houston, TX (US)

(73) Assignee: BOARD OF REGENTS, THE UNIVERSITY OF TEXAS SYSTEM, Austin, TX (US)

(21) Appl. No.: 13/758,728

(22) Filed: Feb. 4, 2013

Related U.S. Application Data
(60) Provisional application No. 61/594,800, filed on Feb. 3, 2012.

(51) Int. Cl. G01N 33/74; A61K 39/395; A61K 31/506; A61K 31/4439; C12Q 1/68; A61K 31/4045

(52) U.S. Cl. CPC G01N 33/74; C12Q 1/6886; A61K 31/4045; A61K 31/506; A61K 31/4439; A61K 39/39558
USPC 424/133.1; 435/7.23; 435/6.11; 514/414; 514/252.18; 514/338

(57) ABSTRACT

Copy number gains detected in tumors and associated with drug sensitivity and resistance in vivo and in vitro can be used as biomarkers to select, predict and monitor drug treatment outcomes in cancer patients treated with tyrosine kinase inhibitors. Methods to identify patients with NSCLC or other malignancies who are more likely to benefit from tyrosine kinase inhibitors such as VEGF or VEGFR inhibitors when used either as monotherapy or in combination with other therapies such as chemotherapy or EGFR inhibitors, and who are in the advanced stages of disease and/or who have undergone adjuvant therapy are also provided herein.

### BACKGROUND OF THE INVENTION

This application claims the benefit of U.S. Provisional Patent Application No. 61/594,800, filed Feb. 3, 2012, the entirety of which is incorporated herein by reference.

This invention was made with government support under Prospect Grant W81XWH-07-1-0306 awarded by the U.S. Department of Defense, Grant W81XWH-06-1-0303 awarded by the U.S. Department of Defense, and Grants 5P50 CA070907-14 and CA-16672 awarded by the National Institutes of Health. The government has certain rights in the invention.

Pursuant to 37 C.F.R. 1.821(c), a sequence listing is submitted herewith as an ASCII compliant text file named “UTSCP1203US.txt”, created on Feb. 4, 2013 and having a size of 4 KB. The content of the aforementioned file is hereby incorporated by reference in its entirety.

### FIELD OF INVENTION

This invention relates generally to cancer treatments with tyrosine kinase inhibitors and more particularly, to methods of predicting cancer treatment outcome for a cancer patient through copy number gain of the KDR, PDGFR, and/or KIT genes.

### BACKGROUND OF THE INVENTION

Successful treatment of cancer has remained elusive despite rapid advances in the field in recent years. One major complicating factor in effective treatment is that conventional diagnostics to characterize tumors offer limited insight as to what types of anti-cancer therapy may be successful for treating any given cancer. In fact, cancer cells exhibit a wide range of resistance/susceptibility to various anti-cancer therapies, thus it has been difficult to predict whether a particular cancer will be resistant or susceptible to any given therapy. The vascular endothelial growth factor receptor-2 (“VEGFR-2”), for example, is known to be present on tumor vascular endothelial cells Inhibitors of VEGFR-2 (KDR) have been developed with the goal of inhibiting tumor angiogenesis in cancer patients. However, there are currently no validated markers for predicting which cancer patients are likely to respond to inhibitors of the VEGF/VEGFR pathway. Likewise, powerful inhibitors of the PDGFR and KIT pathways are being developed for anti-cancer therapy, but it is unclear what types of cancers would be most responsive to such therapies. Methods are needed to help select cancer patients who will experience greater benefit from these inhibitors and who are potentially spared the toxicities of these drugs if they are less likely to benefit.

### SUMMARY OF THE INVENTION

In one embodiment, the present invention provides a method of treating a cancer patient comprising selecting a patient determined to have a cancer with an elevated KDR, PDGFR, or KIT level, and then treating the patient with a VEGF/VEGFR, PDGFR, or KIT pathway inhibitor. In one aspect, a patient determined to have an elevated KDR level may be treated with a VEGF/VEGFR pathway inhibitor. In another aspect, a patient determined to have an elevated PDGFR or KIT level may be treated with either a PDGFR or KIT pathway inhibitor, respectively. In a further aspect, a patient determined to have elevated levels of two or three of KDR, PDGFR, and KIT may be treated with two or more inhibitors of the VEGF/VEGFR, PDGFR, or KIT pathways. In another aspect, a patient determined to have elevated levels of two or three of KDR, PDGFR, and KIT may be treated with an inhibitor that inhibits two, or all three, of the VEGF/VEGFR, PDGFR, and KIT pathways, for example, sunitinib or imatinib.

In one aspect, an elevated KDR, PDGFR, or KIT level may be a gain in the gene copy number of one or more of the genes. In another aspect, an elevated KDR, PDGFR, or KIT level may be an increased mRNA expression. In yet another aspect, an elevated KDR, PDGFR, or KIT level may be an increased protein expression. In certain aspects, an elevated KDR level may be an increased mRNA or protein expression level of a KDR-regulated gene, for example, HIF-1α.

In some preferred embodiments, a cancer patient for treatment or assessment accordingly the embodiments may have a NSCLC or a glioblastoma. In some further aspects, the cancer may be a metastatic cancer or a cancer that has developed resistance to one or more anti-cancer agent. In certain aspects, the cancer patient for treatment according to the embodiments, may receive or have received a secondary therapy such as a surgery or radiotherapy. Thus, in some aspects, a treatment of the embodiments is used as an adjuvant treatment. In other aspects, the cancer patient may be treated with a secondary therapy such as a second drug (e.g., that is not a platinum-based chemotherapeutic agent) or an EGFR inhibitor. A secondary therapy for used according to the embodiments may be applied before, after or essentially simultaneously with a treatment of the embodiments.

Certain aspects of the embodiments concern PDGFR, VEGF/VEGFR and/or KIT pathway inhibitors. For example, VEGF/VEGFR pathway inhibitors may be, without limitation, ramucirumab, sunitinib, bevacizumab, aflibercept, BIBF1120, sorafenib, cediranib, dovitinib, pazopanib, ponatinib, semaxanib, axitinib, PP-121, telatinib, TSU-68, Ki8751, tivozanib, motesanib, regorafenib, vatalanib, or vandetanib. PDGFR pathway inhibitor include, without limitation, imatinib, sunitinib, axitinib, BIBF1120, pazopanib, pnoatinib, MK-2461, dovitinib, crenolanib, PP-121, telatinib, CP 673451, TSU-68, Ki8751, tivozanib, masitinib, motesanib, MEDI-575, or regorafenib. KIT pathway inhibitors include, but are not limited to, imatinib, axitinib, pazopanib, dovitinib, telatinib, Ki8751, tivozanib, masitinib, motesanib, sunitinib, IMG-3G3, nilotinib, dasatinib, regorafenib, or vatalanib.

In another embodiment, the present invention provides a method of predicting the sensitivity of a cancer in a patient to a VEGF/VEGFR, PDGFR, and/or KIT pathway inhibitor comprising obtaining a sample of the cancer and determining the KDR, PDGFR, and/or KIT level in the cells comprising the sample, wherein if the KDR, PDGFR, and/or KIT level is elevated, then the cancer is predicted to be sensitive to a corresponding VEGF/VEGFR, PDGFR, or KIT pathway inhibitors. In certain aspects, a patient predicted to be sensitive to VEGF/VEGFR, PDGFR, or KIT pathway inhibitor may be treated with at least one inhibitor of the VEGF/VEGFR, PDGFR, or KIT pathways. In a further aspect, the method further provides for identifying the patient as having a cancer that is predicted to be sensitive to VEGF/VEGFR, PDGFR, or KIT pathway inhibitors, and reporting whether the cancer is predicted to be sensitive or resistant to the inhibitor. (e.g., by providing written, oral or electronic report). In some aspects, such a report can be provided to the patient, a doctor, a hospital, an insurance company, or a payee.

Another embodiment of the present invention provides a method of monitoring the efficacy of VEGF/VEGFR, PDGFR, or KIT pathway inhibitor treatment on a cancer comprising obtaining samples of the cancer from at least two time points during the course of treatment, determining the KDR, PDGFR, or KIT level in the cells comprising the samples, and comparing the KDR, PDGFR, or KIT levels, wherein the VEGF/VEGFR, PDGFR, or KIT pathway inhibitor treatment is efficacious if the KDR, PDGFR, or KIT level decreases over the course of treatment.

In some aspects, the level of mRNA or protein of a gene regulated by a KDR-regulated gene may be used to represent the KDR level. In one aspect, the KDR-regulated gene is HIF-1α and the gene regulated by HIF-1α is EZH2 or Met.

In another embodiment, the present invention provides a method of predicting the sensitivity of a cancer in a patient to an EGFR inhibitor therapy or platinum-based chemotherapy comprising obtaining a sample of the cancer and determining the KDR level in the sample, wherein if the KDR level is not elevated, then the cancer is predicted to be sensitive to EGFR inhibitors or platinum-based chemotherapy. In a further aspect, the method provides for identifying the patient as having a cancer that is predicted to be sensitive to EGFR inhibitors or platinum-based chemotherapy, and reporting whether the cancer is predicted to be sensitive to EGFR inhibitors or platinum-based chemotherapy. For example, reporting can comprise providing a written, oral or electronic report, e.g., to the patient, a doctor, a hospital, an insurance company, or a payee.

In certain aspects, a patient determined to have a normal or decreased KDR level may be treated with an EGFR inhibitor or platinum-based chemotherapeutic agent. Examples of EGFR inhibitors include, without limitation, erlotinib, gefitinib, afatinib, PF299804, cetuximab, panitumab, zalutumumab, nimotuzumab, matuzumab, OSI-420, Cl-1033, neratinib, WHI-P154, or lapatinib. Platinum-based chemotherapeutic agents for use according to the embodiments include, without limitation, cisplatin or carboplatin.

In one aspect, the patient has not yet undergone an anti-cancer therapy. In another aspect, the patient may have received at least one dose of an anti-cancer therapy, such as an EGFR inhibitor or platinum-based chemotherapeutic agent. Accordingly, I some aspects a method may be a method of monitoring (acquired) resistance to said therapy comprising detecting an elevated KDR level. A patient determined to have acquired resistance to an EGFR inhibitor or platinum-based chemotherapeutic agent may be treated with a VEGF/VEGFR pathway inhibitor.

In a further embodiment, the present invention provides a method of treating a cancer patient comprising determining if the patient has a cancer that is sensitive to VEGF/VEGFR, PDGFR, or KIT pathway inhibitors and treating the patient determined to have a cancer that is sensitive to VEGF/VEGFR, PDGFR, or KIT pathway inhibitors with VEGF/VEGFR, PDGFR, or KIT pathways inhibitors.

In another embodiment, the present invention provides a method of selecting a drug therapy for a cancer patient comprising obtaining a sample of the cancer, determining the KDR, PDGFR, or KIT level in the cells comprising the sample, and selecting a VEGF/VEGFR, PDGFR, or KIT pathway inhibitor for drug therapy if the level determined in (b) is elevated or selecting an EGFR inhibitor platinum-based chemotherapy if the level determined in (b) is not elevated.

The present invention also provides a method of determining a prognosis of a cancer patient comprising obtaining a sample of the patient's cancer and determining the KDR level in the cells comprising the sample, wherein the cancer is determined to have a worse prognosis if the KDR level is determined to be elevated.

The present invention also provides a method of determining a prognosis of a cancer patient comprising obtaining a sample of the patient's cancer and detecting polymorphisms at nucleotides −37 and 1416 in the KDR gene in the cells comprising the sample, wherein the cancer is determined to have a better prognosis if the −37 AG/GG and 1416 AT/TT polymorphisms are present. In one aspect, if the polymorphisms are absent, then an aggressive anticancer therapy may be applied.

Methods of predicting a treatment outcome for a cancer patient, methods of monitoring responsiveness to drug therapy, and methods of selecting drug therapy are provided herein. Also provided are methods to identifying cancer patients who are more likely to benefit from tyrosine kinase inhibitors, such as VEGF or VEGFR inhibitors when used either as monotherapy or in combination with other therapies, such as chemotherapy or EGFR inhibitors, and who are in the advanced stages of disease and/or who have undergone adjuvant therapy. Further provided are methods to identify which patients are more likely to be resistant to tyrosine kinase inhibitors such as EGFR inhibitors. The methods described herein are useful either as a predictive marker prior to starting a drug therapy or as a marker of acquired resistance for patients more likely to benefit from treatment with tyrosine kinase inhibitors, such as VEGF or VEGFR inhibitors, alone or in combination regimens. Moreover, methods are provided that identify patients who would benefit from targeting the PDGFR or KIT pathways, alone or in combination with VEGFR pathway inhibitors, in NSCLC and other malignancies with CNGs in the PDGFR or KIT genes.

Each method described herein includes at least the steps of: providing a biological sample from a cancer patient; determining CNG of at least one of the following genes: KDR, PDGFR, and KIT in the sample, wherein a gene copy number of 4 or greater for the KDR, PDGFR, or KIT gene is considered CNG and predictive of poor treatment outcome; and, when appropriate, administrating a drug or other therapy to the cancer patient based on the CNG of one or more of these genes. In addition, other prognostic methods and/or method steps may be used together with these methods.

Some aspects of the embodiments involve a subject, such as a cancer patient. As used herein a subject or patient can be human or non-human animal subject (e.g., a dog, cat, mouse, horse, etc). In certain aspects, the subject has a cancer, such as an oral cancer, oropharyngeal cancer, nasopharyngeal cancer, respiratory cancer, urogenital cancer, gastrointestinal cancer, central or peripheral nervous system tissue cancer, an endocrine or neuroendocrine cancer or hematopoietic cancer, glioma, sarcoma, carcinoma, lymphoma, melanoma, fibroma, meningioma, brain cancer, oropharyngeal cancer, nasopharyngeal cancer, renal cancer, biliary cancer, pheochromocytoma, pancreatic islet cell cancer, Li-Fraumeni tumors, thyroid cancer, parathyroid cancer, pituitary tumors, adrenal gland tumors, osteogenic sarcoma tumors, neuroendocrine tumors, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer or skin cancer.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-E show that KDR copy number gain (CNG) is correlated with VEGFR-2 protein expression in non-small cell lung carcinomas (NSCLC) tumors. FIG. 1A (copy number gain) and 1B (no copy number gain) are representative examples of KDR copy number examined by fluorescence in situ hybridization (FISH) in NSCLC tissue specimens. Signals represent the KDR gene probe or the internal control probe (magnification ×1000). FIG. 1C (adenocarcinoma) and 1D (squamous cell carcinoma) are representative examples of immunohistochemical expression of VEGFR-2 in NSCLC tissue specimens. VEGFR-2 protein expression was present both in the cytoplasm and membrane of tumor cells (magnification ×200). FIG. 1E shows expression of VEGFR-2 in tumors with KDR CNG compared with lung cancers without CNG. The box-plots depict scores of immunohistochemical (IHC) expression of VEGFR-2 cytoplasm and VEGFR-2 membrane comparing 26 lung cancers having KDR CNG with 26 lung cancers without CNG. In the box plots, bars indicate median score, x indicates mean scores, and dashed lines indicate standard deviation.

FIG. 2A-C show KDR copy number gain (CNG) correlated with microvascular density (MVD) in non-small cell lung carcinomas (NSCLC) tumors. FIG. 2A shows expression of MVD in tumors with KDR CNG compared with lung cancers without CNG. The box-plots depict scores of immunohistochemical assessment of MVD and vessel area (mm²) comparing 26 lung cancers having KDR CNG with 26 lung cancers without CNG. In the box plots, bars indicate median score, x indicates mean scores, and dashed lines indicate standard deviation. FIG. 2B (adenocarcinoma) and 2C (squamous cell carcinoma) are representative examples of immunohistochemical expression of CD34-positive vessels (MVD) (magnification ×200).

FIG. 3 shows KDR copy number gain (CNG) associated with outcome in NSCLC patients treated with adjuvant chemotherapy. Kaplan-Meier curves for overall survival (OS) and recurrence-free survival (RFS) by KDR CNG in NSCLC patients and two subgroups of platinum adjuvant therapy and without adjuvant therapy (E, event; N, total number of cases).

FIG. 4A-E show KDR copy number gain (CNG) and VEGFR-2 expression associated with resistance to cisplatin. FIG. 4A shows the correlation of KDR copy number gain (CNG) with in vitro resistance to cisplatin. NSCLC cell lines demonstrating CNG (≧6 gene copies) showed significantly higher IC₅₀ compared with cell lines without CNG. FIG. 4B shows the correlation between the concentrations of cisplatin required to inhibit NSCLC cell growth (IC₅₀) and VEGFR-2 protein expression levels by reverse phase protein array (RPPA). FIG. 4C shows that siRNA targeting KDR (siKDR) in NSCLC cell line H23 significantly inhibited the expression of VEGFR-2 by Western blot (WB) and KDR mRNA by reverse transcriptase quantitative PCR (RT-qPCR) compared with basal and scrambled control siRNA (Bars: s.d.; *, P<0.05). FIG. 4D shows that knocking down KDR using siRNA decreased the viability of NSCLC cell line H23 exposed to cisplatin by MTS assay (data are graphed as mean percent increase±percent s.d.). Knockdown of KDR in H23 cells caused a 1.9-fold decrease in the cisplatin IC₅₀ (53 versus 97.9 μmol/L in siKDR knockdown H23 cells versus untransfected cells; P<0.05) and a 3.5-fold decrease in the carboplatin IC₅₀ (27.9 versus 97 μmol/L in siKDR knockdown H23 cells versus non-transfected cells; P<0.05). FIG. 4E shows the migration of NSCLC cell line H23 by Boyden chamber assay (left) was inhibited by knocking down KDR using KDR in cells with and without stimulation with VEGF (Bars: s.d.; *P<0.05: **P<0.003). The right panel shows the quantification of the migration assay of NSCLC cell lines before and after knocking down KDR using siKDR in cells with and without stimulation with VEGF showed decreased migration in H23 cells (6-9 KDR copies).

FIG. 5A-E show KDR copy number gain (CNG) correlated with HIF-1α expression in NSCLC cell lines and tumor tissue specimens. FIG. 5A shows HIF-1α protein expression determined by ELISA correlated with KDR CNG in a series of NSCLC cell lines (Bars: s.d.; cell lines with CNG 6-9 copies versus 3-5 copies and no CNG, *P<0.02). FIG. 5B shows HIF-1α expression by ELISA was markedly inhibited by knocking down KDR using siKDR in the NSCLC H23 cell line with and without stimulation with VEGF (Bars: s.d.; *P<0.01). FIG. 5C shows expression of nuclear HIF-1α in tumors with KDR CNG compared with lung cancers without CNG. The box-plots depict scores of immunohistochemical (IHC) expression of nuclear HIF-1α comparing 22 lung cancers having KDR CNG with 25 lung cancers without CNG. In the box plots, bars indicate median score, x indicates mean scores, and dashed lines indicate standard deviation. FIG. 5D (adenocarcinoma) and 5E (squamous cell carcinoma) are representative examples of low (FIG. 5D) and high (FIG. 5E) IHC expression of HIF-1α in NSCLC tissue specimens (magnification ×200). Arrows, positive nuclear HIF-1α immunostaining.

FIG. 6 shows VEGFR inhibitor, sunitinib, inhibits cell migration in H23 cells which harbors VEGFR CNGs. Imatinib, which targets BCL/ABL, Kit, and PDGFR, does not inhibit cellular migration. In contrast, the VEGFR inhibitor, sunitinib, has no effect on migration of A549 cells, which do not have amplification of VEGFR.

FIG. 7A-C show that HIF-1α levels are decreased by VEGFR inhibition in VEGFR amplified cells. FIG. 7A shows higher levels of HIF-1α in cell lines with VEGFR CNGs compared to those without. FIG. 7B shows a statistically significant decrease in HIF-1α levels in H23 cells (KDR CNG+) treated with the VEGFR inhibitor sunitinib. FIG. 7C shows no change in HIF-1α levels was detected in A549 cells, which do not contain VEGF CNGs.

FIG. 8 shows that VEGFR pathway inhibition with bevacizumab decreases HIF-1α-regulated proteins, including EZH2, Met, and phosphorylated Met, in H23 and Calu1 cells, which have VEGFR CNGs. Two VEGFR amplified cell lines, H23 and Calu1, were treated with the VEGFR pathway inhibitor bevacizumab and evaluated for changes in proteins regulated by HIF-1α. Multiple HIF-1α-regulated proteins were decreased in the presence of bevacizumab, including EZH2, Met, and phosphorylated Met.

FIG. 9 shows the Kaplan-Meier curves for overall survival (OS) by genotypes of two KDR single nucleotide polymorphisms in adenocarcinoma and squamous cell carcinoma of lung (E, event; N, total number of cases).

FIG. 10 A-C show that VEGFR TKIs inhibit cell migration in KDR amplified cell lines. Each cell line was tested with or without VEGF (50 ng/mL) and with or without AZD2171, sunitinib, and imatinib (bars: s.d.; *P<0.05 vs. control; #P<0.05 vs. VEGF alone). FIG. 10A shows the quantification for the number of migrating cells relative to control for the Calu-1 cell line. FIG. 10B shows the quantification for the number of migrating cells relative to control for the HCC461 cell line. FIG. 10C shows the quantification for the number of migrating cells relative to control for the H1993 cell line.

FIG. 11 shows the effect of VEGFR TKIs on tumor cell secretion of cytokines H23 tumor cells were treated with control media or media containing the VEGFR TKI sunitinib (1 μM) for 24 hours. Conditioned media was collected and cytokine levels (VEGF, PDGF, IL-8, HGF, and FGF2) were assessed by ELISA assay. Imatinib was used as a negative control.

FIG. 12 shows that KDR copy number gain was associated with increased levels of EGFR and greater expression of mTOR pathway components mTOR and p70s6K. KDR copy number was compared with expression of a broad panel of proteins screened by reverse phase protein array for various cell lines.

FIG. 13 shows that VEGF increased tumor cell survival in the presence of erloninib and axitinib reversed the effect. HCC827 cells, which harbor the EGFR activating mutation, were treated with VEGF and with or without the VEGFR TKI axitinib. After 24 hours, increasing concentrations of erlotinib were added to the cells.

FIG. 14 shows that patients with EGFR-driven cancer that were treated with erlotinib did worse when they had high vs. low levels of KDR (P=0.001). This analysis was performed on clinical specimens from the BATTLE clinical trial.

### DETAILED DESCRIPTION OF THE INVENTION

Methods and compositions for predicting disease outcome for cancer patients treated with tyrosine kinase inhibitors are provided herein. Copy number gain (“CNG”) of certain genes can serve as biomarkers for predicting cancer treatment outcome of kinase inhibitors, especially inhibitors of vascular endothelial growth factor receptor (“VEGFR”), epidermal growth factor receptor (“EGFR”), platelet-derived growth factor receptor (“PDGFR”), and kinase insert domain receptor (“KIT”). Specifically, the copy number gain of KDR, PDGFR, and KIT genes, alone or in combination with each other, can be used to predict whether a patient may benefit from one or more tyrosine kinase inhibitor drug therapies.

As such, methods of predicting a treatment outcome for a cancer patient, methods of monitoring responsiveness to drug therapy, methods of selecting drug therapy and methods of identifying patients with NSCLC or other malignancies who are more likely to benefit from VEGF, VEGFR, or EGFR inhibitors, and/or inhibitors of the PDGFR and/or KIT pathways are provided herein. Each method includes at least the steps of: (a) providing a biological sample from a cancer patient; (b) determining CNG, wherein a gene copy number of greater than 4 for either the KDR, PDGFR, or KIT gene is considered CNG and predictive of poor treatment outcome; and (c) administrating a drug or other therapy to the cancer patient based on the CNG of one or more genes.

Deregulated kinase activity is a frequent cause of disease, particularly cancer, where kinases regulate many aspects that control cell growth, movement, and death. Many of the genetic defects can identify the key components of signaling pathways responsible for proliferation and differentiation. One class of kinases that are frequently deregulated in cancer are receptor tyrosine kinases (“RTKs”) involved in signal transduction. In general, RTKs are monomeric surface receptors that dimerize upon activation. RTKs have an extracellular binding domain, a transmembrane domain, and an intracellular kinase domain. Ligand binding to the extracellular domain induces dimerization of the surface receptor, which in turn induces phosphorylation of tyrosine residues within an “activation loop” of the intracellular kinase domain.

Tumor growth is critically dependent on neovascularization (Folkman, 1971). The ligand vascular endothelial growth factor (“VEGF”) is an endothelial cell mitogen that is a specific mediator of angiogenesis and has two identified tyrosine kinase receptors, VEGF receptor-1 and -2 (Fidler et al., 1994; Waltenberger et al., 1994; Ferrara et al., 1997; Hanahan et al., 2011).

VEGFR-2 coded by the gene FLK-I (located in 4q12) is the predominant mediator of vascular endothelial growth factor-stimulated endothelial cell functions, including cell migration, proliferation, survival, and enhancement of vascular permeability. (Terman et al., 1991; Bernatchez et al., 1999). VEGFR-2 exhibits robust protein-tyrosine kinase activity in response to the binding of vascular endothelial growth factor (“VEGF”) ligand (Waltenberger et al., 1994).

In human epithelial tumors, including lung, vascular endothelial growth factor-2 (“VEGFR-2” or noted as“VEGFR2”) is expressed in malignant cells as well as in the endothelial cell of tumor vasculature. Furthermore, in non-small cell lung carcinoma (“NSCLC”), VEGFR-2 is overexpressed in malignant cells of tumor tissues and associated with a poor outcome (Ishii et al., 2004; Ludovini et al., 2004; Seto et al., 2006; Carrillo de Santa Pau et al., 2009; Donnem et al., 2009). Moreover, tumor cell expression of VEGFR-1 can drive tumor cell invasiveness and promote hypoxia-independent upregulation of hypoxia inducible factor-1α (HIF-1α) (Nilsson et al., 2010; Roybal et al., 2010). EGFR (“epidermal growth factor receptor”) is a cell surface receptor activated by binding of its specific ligands, including epidermal growth factor and transforming growth factor α (“TGFα”). Upon activation by its growth factor ligands, EGFR undergoes a transition from an inactive monomeric form to an active homodimer. In addition to forming homodimers, EGFR may pair with another member of the ErbB receptor family, such as ErbB2/Her2/neu, to create an activated heterodimer. Mutations of EGFR or amplification can lead to its constant activation, resulting in uncontrolled cell division, a predisposition of cancer. Consequently, mutations and amplifications of EGFR have been identified in several types of cancer, including lung cancer, glioblastoma multiforme, and renal cancer, and have been associated with improved clinical benefit for patients receiving EGFR inhibitors, such as erlotinib or gefitinib (Paez et al., 2004; Lynch et al., 2004; Mok et al., 2009). While these patients may have improved responses to EGFR inhibitors, tumors eventually become resistant. One mechanism for developing resistance is through amplification of the MET receptor tyrosine kinase (Engelman et al., 2007), which provides a “bypass” for activating signaling pathways in the cancer cell even when EGFR is blocked. There is a need to identify other potential “bypass” pathways that can be blocked with drug treatment to prevent or overcome EGFR inhibitor drug resistance.

Generally, growth factors are polypeptides involved in the regulation of cell growth and differentiation, such as, during embryonal development, in wound healing, in hematopoiesis, in the immune response, as well as in several adverse reactions, including malignancies. As such, platelet-derived growth factor (“PDGF”) was originally found to promote cell growth and division, particularly in fibroblasts and smooth muscle cells. Subsequently, however, PDGF has been shown to be synthesized by a large number of different normal cells as well as transformed cell types. PDGF acts by binding to the PDGF receptor tyrosine kinases (PDGFRs), including PDGFR-alpha. PDGFRs are currently known to play a significant role in blood vessel formation or angiogenesis and have been implicated in promoting tumor growth in different types of cancers, including lung cancer (Ballas et al., 2011). There are a number of drugs that block PDGFRs, including imatinib and sunitinib. There are currently no validated markers for identifying which patients are likely to benefit from these drugs.

The c-Kit protein is an RTK and is often designated as KIT in the literature together with a variety of other possible variations, including, but not limited to, c-kit, kit, KIT, c-Kit, and c-KIT. Likewise, the gene encoding c-Kit is often designated in the literature as kit or c-kit. Moreover, as with protein designations, the terms c-kit, c-KIT, KIT, kit, and c-Kit can be associated with the gene that encodes the protein and variations thereof. Therefore, as used herein, any one of a number of possible variations of the term designating the KIT protein and the gene encoding this protein can and may be used interchangeably herein.

Furthermore, the protein-tyrosine kinase KIT is also the transmembrane receptor for stem cell factor (SCF). SCF, also known as “steel factor,” “c-kit ligand,” or “CD117” is a polypeptide that activates bone marrow precursors of a number of blood cells. However, SCF's receptor (c-Kit) is also present on tumor cells including lung cancer cells and can promote the survival and invasiveness of cancer cells (Kijima et al., 2002). There are a number of drugs in clinical use or development that inhibit KIT, including imatinib and sunitinib.

Kinase insert domain receptor (“KDR”), a VEGF receptor, is a type III receptor tyrosine kinase and is also known as vascular endothelial growth factor receptor 2 (“VEGFR-2”). KDR also refers to the human gene encoding the receptor. KDR has also been designated as CD309 (cluster of differentiation 309). KDR is also known as Flk1 (Fetal Liver Kinase 1). As described herein, VEGFR-2/KDR is a known endothelial target also expressed in NSCLC tumor cells. As described in Example 1 below, the association between alterations in the KDR gene and clinical outcome in patients with resected NSCLC (n=248) was investigated. KDR copy number gains (CNGs), measured by quantitative PCR and fluorescence in situ hybridization, were detected in 32% of tumors and were associated with significantly higher KDR protein and higher microvessel density than tumors without CNGs. KDR CNGs were also associated with significantly increased risk of death (HR=5.16; P=0.003) in patients receiving adjuvant platinum-based chemotherapy, but no differences were observed in patients not receiving adjuvant therapy. To investigate potential mechanisms for these associations, NSCLC cell lines were assessed and it was found that KDR CNGs were significantly associated with in vitro resistance to platinum chemotherapy, as well as increased levels of nuclear HIF-1α in both NSCLC tumor specimens and cell lines (α is also noted sometimes herein as alpha and β as beta, etc). Furthermore, KDR knockdown experiments using small interfering RNA reduced platinum resistance, cell migration, and HIF-1α levels in cells bearing KDR CNGs, providing evidence for direct involvement of KDR. No KDR mutations were detected in exons 7, 11, and 21 by PCR-based sequencing; however, two variant genotypes SNPs were associated with favorable OS in patients with adenocarcinoma. Cells with KDR CNG were also more sensitive to inhibition with drugs inhibiting VEGFR-2, such as sunitinib, and cells with KDR CNG became more resistant to EGFR inhibitors after treatment with VEGF. Based on this, KDR CNG can promote a more malignant phenotype, including increased chemoresistance, angiogenesis, and HIF-1α levels. Furthermore, KDR CNG can be a useful biomarker for identifying patients at high risk for recurrence after adjuvant therapy, or that are more likely to be resistant to chemotherapy, two groups that may benefit from VEGF or VEGFR-2 blockade. KDR CNG may also identify patients more likely to benefit from VEGF or VEGFR-2 blockade, or that might be resistant to EGFR inhibitors.

The KDR gene is adjacent to PFGFR and KIT, receptor tyrosine kinase (“RTK”) genes that are often co-amplified as part of an amplicon. Multiple RTKs can interact to drive the malignant phenotype in different cancers (Nilsson et al., 2010; Xu et al., 2010). Hence, the assessment of CNGs of one or more of the three RTKs in the amplicon (KDR, PDGFR, and KIT) may be useful to predict whether a patient may benefit from drugs targeting one or more of these RTKs, alone or in combination.

Selective inhibitors are defined as those that have an IC₅₀ value against the target kinase that is less than about 1/10, and preferably less than about 1/20 the IC₅₀ value against a non-target enzyme. In addition, inhibitors that are selective for a specific target kinase are defined as having a selectivity ratio of at least about 10, and more preferably at least about 40, of target inhibition over off-target inhibition. Bevacizumab is an example of a selective VEGF/VEGFR inhibitor used in the present invention. Dual inhibitors are defined as those that inhibit two or more targets in a selective manner relative to non-target enzymes. Imatinib is an example of a dual inhibitor used in the present invention.

As provided herein, copy number gain (“CNG,” or as referred to in the plural, “CNGs”) of certain genes are associated with increased likelihood of relapse in cancer patients receiving adjuvant therapy and/or chemotherapy. Specifically, the CNG of genes, such as KDR, PDGFR, and KIT, can serve as biomarkers (also referred to herein as “markers”) alone or in combination with other biomarkers. These biomarkers can be used to predict treatment outcomes in cancer patients who have received adjuvant therapy and patients treated with different drugs. More specifically, CNG of the KDR, PDGFR, and KIT genes can, each alone or in combination, serve as markers for predicting treatment outcomes for patients being treated with drug therapies including, but not limited to, VEGFR2, EGFR, PDGFR, and KIT inhibitors and chemotherapy. As used herein, a CNG is a gene copy number of 4 or greater. Patients with CNG will benefit from treatments with tyrosine kinase inhibitors or other drugs targeting the VEGFR, PDGFR, or KIT pathways (e.g., an antibody to VEGF).

As noted herein, each of the methods described comprises the step of: (a) providing a biological sample from a cancer patient; (b) determining CNG for at least one of the following genes: KDR, PDGFR, and KIT in the sample, wherein a gene copy number of at least 4 for either of the KDR, PDGFR, or KIT genes is predictive of poor drug treatment outcome; and, (c) if appropriate, administrating a drug or other therapy to the cancer patient based on the prediction obtained. In addition, other prognostic method steps may be used together with these methods. For example, protein expression in the patient sample may also be determined, the proteins including VEGFR2 and others, such as soluble VEGFR2 (a truncated version of VEGFR2), VEGFR1, VEGFR3, HIF-1α, EGFR, PDGFR, EZH2, and KIT.

For the methods provided herein, the term biological samples refers to any biological sample obtained from an individual, including body fluids, body tissue, cells, or other sources known to those skilled in the art. Also, the terms “sample” and “biological sample” are used interchangeably herein. For example, a sample can be a tissue sample, such as a peripheral blood sample that contains circulating tumor cells, or a lung tumor tissue biopsy or resection. Other samples may include a thin layer cytological sample, a fine needle aspirate sample, a lung wash sample, a pleural effusion sample, a fresh frozen tissue sample, a paraffin embedded tissue sample, or an extract or processed sample produced from any of a peripheral blood sample. Body fluids, such as lymph, sera, whole fresh blood, peripheral blood mononuclear cells, frozen whole blood, plasma (including fresh or frozen), urine, saliva, semen, synovial fluid, and spinal fluid are also suitable as biological samples. Samples can further include breast tissue, renal tissue, colonic tissue, brain tissue, muscle tissue, synovial tissue, skin, hair follicle, bone marrow, and tumor tissue.

The genetic biomarkers (also referred to herein as a “biomarker” or “marker”) provided herein can be detected using any method known in the art. For example, a biological sample obtained from the patient can be analyzed via in situ hybridization, such as fluorescent in situ hybridization (FISH), having fluorescently labeled nucleic acid probes or fluorescently labeled probes comprising nucleic acid analogs can be used to determine the CNGs. Alternatively, polymerase chain reaction, a nucleic acid sequencing assay, or a nucleic acid microarray assay may be used.

In general, in situ hybridization includes the steps of fixing a biological sample, hybridizing one or more chromosomal probes to target DNA contained within the fixed sample, washing to remove non-specifically bound probe, and detecting the hybridized probe. The in situ hybridization can also be carried out with the specimen cells from the biological sample in liquid suspension, followed by detection by flow cytometry. A FISH assay can be used to evaluate chromosomal copy number abnormalities in a biological sample from a patient. FISH probes for use in the methods may comprise a pair of probes specific to gene or chromosomal locus, which may include any portion of the sequence encoding the gene.

The term “patient” means all mammals including humans. Examples of patients include humans, cows, dogs, cats, goats, sheep, pigs, and rabbits. Preferably, the patient is a human.

A “disorder” or “disease” is any condition that would benefit from treatment with a substance/molecule or method of the invention. This includes chronic and acute disorders or diseases including those pathological conditions that predispose the mammal to the disorder in question. Furthermore, non-limiting examples of disorders to be treated herein include malignant and benign tumors; non-leukemias and lymphoid malignancies; neuronal, glial, astrocytal, hypothalamic, and other glandular, macrophagal, epithelial, stromal, and blastocoelic disorders; and inflammatory, immunologic, and other angiogenic disorders.

The methods described herein are useful in treating cancer, particularly, metastatic disease and after adjuvant therapy, such as surgery or radiotherapy. Generally, the terms “cancer” and “cancerous” refer to or describe the physiological condition in mammals that is typically characterized by unregulated cell growth. More specifically, cancers that are treated using any one or more tyrosine kinase inhibitors, other drugs blocking the receptors or their ligands, or variants thereof, and in connection with the methods provided herein include, but are not limited to, carcinoma, lymphoma, blastoma, sarcoma, leukemia, squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer and gastrointestinal stromal cancer), pancreatic cancer, glioblastoma, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, melanoma, superficial spreading melanoma, lentigo maligna melanoma, acral lentiginous melanomas, nodular melanomas, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

An effective response of a patient or a patient's “responsiveness” to treatment refers to the clinical or therapeutic benefit imparted to a patient at risk for, or suffering from, a disease or disorder. Such benefit may include cellular or biological responses, a complete response, a partial response, a stable disease (without progression or relapse), or a response with a later relapse. For example, an effective response can be reduced tumor size or progression-free survival in a patient diagnosed with cancer.

Treatment outcomes can be predicted, monitored and selected and/or patients benefiting from such treatments can be identified via the methods described herein for the tyrosine kinase inhibitors of the VEGF/VEGFR pathway or related pathways, including VEGFR inhibitors, drugs targeting VEGF or other VEGF family ligands, such as VEGF-C, EGFR inhibitors, PDGFR inhibitors, and KIT inhibitors. As such, VEGFR2 inhibitors useful in identifying patients and predicting, monitoring, or selecting treatments include, but are not limited to sunitinib, sorafenib, axitinib, vandetanib, cediranib, bevacizumab, ramucirumab, BIBF1120, aflibercept, tivozanib, semaxanib, dovitinib, PP-121, telatinib, TSU-68, Ki8751, motesanib, regorafenib, vatalanib, ponatinib, and pazopanib. Preferred inhibitors are sunitinib and bevacizumab.

Likewise, many therapeutic approaches are aimed at the EGFR. Cetuximab and panitumab are examples of monoclonal antibodies. However, the former is of the IgG1 type, the latter of the IgG2 type. Other monoclonal antibodies directed towards blocking EGFR are zalutumumab, nimotuzumab, and matuzumab. These monoclonal antibodies block the extracellular ligand binding domain. With the binding site blocked, signal molecules can no longer attach there and activate the tyrosine kinase. Furthermore, additional EGFR inhibitors useful in connection with the methods described herein include, but are not limited to, erlotinib gefitinib, afatinib, lapatinib, neratinib, WHI-P154, OSI-420, Cl-1033, and PF299804. Currently, the identification of EGFR as an oncongene has led to the development of anticancer therapeutics directed against EGFR, including, but not limited to, gefitinib and erlotinib for lung cancer, and cetuximab for colon cancer.

Tyrosine kinases are a subgroup of the larger class of protein kinases. Fundamentally, a protein kinase is an enzyme that modifies a protein by chemically adding phosphate groups via phosphorylation. Such modification often results in a functional change to the target protein or substrate by changing the enzyme activity, cellular location, or association with other proteins. Chemically, the kinase removes a phosphate group from ATP and covalently attaches it to one of three amino acids (serine, threonine, or tyrosine) that have a free hydroxyl group. Most kinases act on both serine and threonine, and certain others, tyrosine. There are also a number of kinases that act on all three of these amino acids. Generally, kinases are enzymes known to regulate the majority of cellular pathways, especially pathways involved in signal transduction or the transmission of signals within a cell. Because protein kinases have profound effects on a cell, kinase activity is highly regulated. Kinases can be turned on or off by phosphorylation (sometimes by the kinase itself through cis-phosphorylation/autophosphorylation) and by binding to activator proteins, inhibitor proteins, or small molecules.

Small molecules can inhibit the EGFR tyrosine kinase, which is on the cytoplasmic side of the receptor. Without kinase activity, EGFR is unable to activate itself, which is a prerequisite for binding of downstream adaptor proteins. Ostensibly, by halting the signaling cascade in cells that rely on this pathway for growth, tumor proliferation and migration is diminished. Gefitinib, erlotinib, lapatinib (mixed EGFR and ERBB2 inhibitor), afatinib, and PF299804 are examples of small molecule kinase inhibitors. Patients have been divided into EGFR-positive and EGFR-negative based upon whether a tissue test shows a mutation. EGFR-positive patients have shown an impressive 60% response rate, which exceeds the response rate for conventional chemotherapy.

PDGFR inhibitors useful in connection with the methods described herein include, but are not limited to, imatinib, sunitinib, axitinib, BIBF1120 (Vargatef), pazopanib, ponatinib, MK-2461, dovitinib, crenolanib, PP-121, telatinib, CP 673451, TSU-68, Ki8751, tivozanib, masitinib, motesanib, regorafenib, and MEDI-575. Preferred inhibitors are imatinib and sunitinib.

KIT inhibitors useful in the methods described herein include, but are not limited to imatinib, sunitinib, dasatanib, IMC-3G3, pazopanib, dovitinib, telatinib, Ki8751, tivozanib, masitinib, motesanib, regorafenib, vatalanib, and nilotinib. Preferred inhibitors are imatinib and sunitinib.

A. Detection of Copy Number Gain

As applied herein, CNG is when the gene copy number is 4 or greater. Hybridization-based assays include, but are not limited to, traditional “direct probe” methods, such as Southern blots or in situ hybridization (e.g., FISH), and comparative probe methods, such as Comparative Genomic Hybridization (CGH). The methods can be used in a wide variety of formats including, but not limited to substrate (e.g., membrane or glass)-bound methods or array-based approaches as described below.

Generally, in situ hybridization includes the steps of: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization; and (5) detection of the hybridized nucleic acid fragments. The reagents used in each of these steps and the conditions for use vary depending on the particular application. The probes are typically labeled, e.g., with radioisotopes or fluorescent reporters. The preferred size range is from about 200 bp to about 1000 bp, more preferably between about 400 and about 800 bp for double stranded, nick translated nucleic acids.

In comparative genomic hybridization methods, a first collection of (sample) nucleic acids (e.g., from a possible tumor) is labeled with a first label, while a second collection of (control) nucleic acids (e.g., from a healthy cell/tissue) is labeled with a second label. The ratio of hybridization of the nucleic acids is determined by the ratio of the two (first and second) labels binding to each fiber in the array. Where there are chromosomal deletions or multiplications, differences in the ratio of the signals from the two labels will be detected and the ratio will provide a measure of the copy number.

A variety of other nucleic acid hybridization formats are known to those skilled in the art. For example, common formats include sandwich assays and competition or displacement assays. The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods include the nucleic acid sequence based amplification.

Amplification-Based Assays

Amplification-based assays could be used to measure CNGs. In such amplification-based assays, the nucleic acid sequences act as a template in an amplification reaction (e.g., Polymerase Chain Reaction (“PCR”)). In a quantitative amplification, the amount of amplification product will be proportional to the amount of template in the original sample. Comparison to appropriate (e.g., healthy tissue) controls provides a measure of the copy number of the desired target nucleic acid sequence. Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990). Other suitable amplification methods include, but are not limited to, ligase chain reaction (LCR), transcription amplification, and self-sustained sequence replication.

B. Detection of Expressed Protein

A polypeptide can be detected and quantified by any of a number of means known to those of skill in the art, including analytic biochemical methods, such as electrophoresis, capillary electrophoresis, high performance liquid chromatography (“HPLC”), thin layer chromatography (“TLC”), hyperdiffusion chromatography, and the like, or various immunological methods, such as fluid or gel precipitation reactions, immunodiffusion (single or double), immunoelectrophoresis, radioimmunoassay (“RIA”), enzyme-linked immunosorbent assay (“ELISA”), immunofluorescent assays, western blotting, and the like.

As provided in Example 1 below, a high frequency of KDR CNG (32%) in both major histology types of NSCLC, adenocarcinoma and squamous cell carcinoma, by qPCR, has been confirmed in a subset of cases by FISH in lung cancer. Conversely, mutations of KDR were rarely detected in NSCLC cell lines and not detected in tumor specimens; however, two variant genotype SNPs (1416 AT/TT and −37 AG/GG) were associated with favorable OS in patients with adenocarcinoma. KDR CNGs in tumors were associated with significantly higher KDR protein expression and higher microvessel density than tumors without CNGs. Notably, KDR CNG predicted worse overall survival in patients who received platinum adjuvant therapy but not in untreated patients. To investigate potential mechanisms for these associations NSCLC cell lines were assessed and it was found that KDR CNGs were significantly associated with in vitro resistance to platinum chemotherapy, as well as increased levels of nuclear HIF-1α in both NSCLC tumor specimens and cell lines. Furthermore, KDR knockdown experiments using small interfering RNA reduced platinum resistance, cell migration, and HIF-1α levels in cells bearing KDR CNGs, providing evidence for direct involvement of KDR. Tumor cell KDR CNGs promote more malignant phenotypes, including increased chemoresistance, angiogenesis, and HIF-1α levels. Furthermore, KDR CNG in malignant cells represents a predictive marker of worse outcome in patients with surgically resected NSCLC treated with platinum adjuvant chemotherapy.

Also described in Example 1, tumors with KDR CNG in the malignant cells showed significantly higher VEGFR-2 protein expression in the cytoplasm and membrane of those cells, as well as higher MVD and larger vessel areas in the tumor stroma, compared with tumors lacking the KDR CNG. One possible explanation for this association is that tumor cell VEGFR-2 binds circulating VEGF, increasing local concentrations of the ligand which turn increases angiogenesis through effects on tumor endothelium. Another possible explanation is that VEGFR-2-overexpressing lung cancer cells may express increased levels of VEGF and other pro-angiogenic factors via upregulation of HIF-1α, which in turn could promote autocrine or paracrine signaling that further increases expression. However, these mechanisms are not mutually exclusive. Furthermore, correlations between KDR CNG and higher expression of HIF-1α in NSCLC cell lines and tumor specimens support the latter hypothesis. Moreover, it has been demonstrated that activation of several receptor tyrosine kinases (RTKs), including RET, VEGFR-1, EGFR, and PDGFR, increases HIF-1α levels in a cell-specific manner in tumors (Nilsson et al., 2010; Hirami et al., 2004; Phillips et al., 2005). Therefore, these data represent the first evidence suggesting that VEGFR-2 may be another RTK that plays a role in increasing the levels of HIF-1α expression in cancer.

As further provided in the study, KDR CNG in malignant cells predicted a worse outcome of NSCLC patients receiving platinum adjuvant chemotherapy after surgical resection with curative intent, but was not predictive in patients without adjuvant therapy. As such, KDR CNG represents a biomarker for predicting resistance to adjuvant platinum-based chemotherapy in NSCLC patients and other cancer patients. In the study, VEGFR-2 knockdown reduced chemoresistance and cell migration, and lowered HIF-1α levels, using in vitro NSCLC models. Hence, the VEGFR-2 blockade may sensitize tumors bearing KDR CNGs to chemotherapy through direct effects on the tumor cells themselves, in addition to its effect on tumor endothelial cells. KDR CNGs can, therefore, identify a group of NSCLC patients that would receive greater relative benefit from combinations of VEGF pathway inhibitors with chemotherapy, or VEGF pathway inhibitors alone, than patients lacking KDR CNGs.

That KDR CNG by SNP array and higher levels of VEGFR-2 expression by RPPA in a large series of NSCLC cell lines correlated significantly with in vitro resistance to platinum dugs (cisplatin for KDR CNG, and cisplatin and carboplatin for VEGFR-2 expression) provides support to the reported clinical observation. The increased sensitivity of the NSCLC cell lines having KDR CNG to in vitro treatment with cisplatin or carboplatin after inhibition of KDR mRNA and protein expressions further supports the concept that KDR CNG may promote platinum resistance in NSCLC. Although the exact mechanism needs to be elucidated, it is postulated that the increased expression of HIF-1α may be induced by KDR CNG, and subsequent VEGFR-2 expression, in malignant NSCLC cells may explain increased platinum resistance in NSCLC. Interestingly, HIF-1α has been previously associated with chemoresistance in NSCLC and other solid tumors (Mi et al., 2008; Koukourakis et al., 2002; Tan et al., 2009).

In NSCLC, chemoresistance to doxorubicin in cell lines A549 has been shown to be partially mediated by enhancement of HIF-1α mediated angiogenesis (Mi et al., 2008). In addition, in the same NSCLC cell line, HIF-1α overexpression-associated chemoresistance might be due to the negative regulation of cyclin D1, leading to the decrease of the cells in S phase and subsequent resistance of cancer cells to antimetabolic cell cycle-specific agents (Wen et al., 2010).

The variant genotypes of KDR SNPs 1416 (AT/TT) and −37 (AG/GG) associated with a favorable OS in the multivariate analysis. This is the first report showing association between KDR SNP genotypes and prognosis in lung cancer. In breast cancer patients the KDR SNP 1416 A/T genotypic variant was associated with the expression of progesterone receptors, and its presence suggested a better prognosis for carriers of the T allele (Forsti et al., 2007). Interestingly, the KDR SNP 1416 A/T (Q472H), a non-synonymous coding polymorphism, is located in the fifth immunoglobulin-like domain within the extracellular region of VEGFR-2 and is important for preventing VEGF-independent receptor dimerization and signal transduction (Tao et al., 2001). The other prognostic KDR SNP in lung adenocarcinoma patients, SNP-37AG/GG is located in intron 11 within the protein kinase domain and has not been associated with any specific protein functional effect. These findings indicate that KDR CNG was frequently detected in NSCLC tumors and associated with platinum resistance in vivo and in vitro, and may be a useful biomarker for identifying patients at high risk for recurrence after adjuvant therapy, a group that may benefit from VEGFR-2 blockade. In addition, KDR SNP genotypes correlate with outcome in patients with surgically resected NSCLC tumors. This is the first report to demonstrate the clinical importance of CNG and genetic variations of KDR in NSCLC.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example I

The objective of this study was to characterize the molecular abnormalities of VEGFR-2 in epithelial malignant cells of NSCLC major histology types, adenocarcinoma and squamous cell carcinoma, and correlate with patients' clinical characteristics. The inventors studied KDR copy number gain (“CNG”), mutation, and genetic variations in malignant cells of surgically resected NSCLC tumor tissues and correlated the results with pathological features in NSCLC patients' tumors and with their platinum adjuvant treatments and outcomes. In addition, using a series of NSCLC cell lines and tissue specimens, the inventors investigated molecular mechanisms associated with KDR CNG in resistance to platinum, particularly the potential role of HIF-1, a key regulator of angiogenesis in malignant tumors.

Material and Methods

NSCLC Tumor Specimens and Cell Lines.

Archived frozen and formalin-fixed and paraffin-embedded (FFPE) tissues from NSCLC patients who were surgically resected with curative intent were obtained. Tissues were selected from the Lung Cancer Specialized Program of Research Excellence (SPORE) tissue bank at The University of Texas M. D. Anderson Cancer Center (Houston, Tex.). The tissue banking and the study were approved by the Institutional Review Board. The tumors were classified using the 2004 World Health Organization (WHO) classification system (Mountain, 1997). Two hundred forty-eight NSCLC specimens (159 adenocarcinomas and 89 squamous cell carcinomas) were randomly selected to test KDR abnormalities. Detailed clinical and pathologic information of the cases is presented in Table 1. The median follow-up of the patients was 3.53 years for those who were censored. All NSCLC cell lines utilized were authenticated by DNA-fingerprinting.

TABLE 1
Clinicopathologic characteristics of non-small lung carcinoma examined for
KDR abnormalities.

| | All Cases | Cases Tested For | Cases Tested For |
|---|---|---|---|
| | Tested | Copy Gain | SNPs± |
| | (N = 248) | (N = 139) | (N = 200) |
| Characteristic | Number (%) | Number (%) | Number (%) |
| Mean Age in Years | 64.6 (26.4-86.9) | 64.9 (32.2-84) | 63.97 (26.4-86.9) |
| (range) | | | |
| Gender | | | |
| Female | 110 (44) | 57 (41) | 88 (44) |
| Male | 138 (56) | 82 (59) | 112 (56) |
| Tumor Histology | | | |
| Adenocarcinoma | 159 (64) | 85 (61) | 127 (64) |
| Squamous cell | 89 (36) | 54 (39) | 73 (36) |
| carcinoma | | | |
| TNM Pathology Stage | | | |
| I | 120 (49) | 70 (51) | 86 (43) |
| II | 50 (20) | 28 (20) | 40 (20) |
| III | 72 (29) | 39 (28) | 63 (34) |
| IV | 6 (2) | 2 (1) | 6 (3) |
| Smoking status± | | | |
| Current | 102 (41) | 52 (37) | 89 (45) |
| Former | 108 (44) | 64 (46) | 82 (41) |
| Never | 38 (15) | 23 (17) | 29 (14) |
| Neoadjuvant therapy+ | | | |
| No | 181 (73) | 115 (83) | 133 (67) |
| Yes | 62 (27) | 24 (17) | 67 (33) |
| Adjuvant therapy+ | | | |
| No | 138 (56) | 69 (50 | 90 (45) |
| Yes | 110 (44) | 70 (50) | 110 (55 |

* SNP, Single Nucleotide Polymorphism.
±Patients who had smoked at least 100 cigarettes in their lifetime were defined as ever smokers, and smokers who quit smoking at least 12 months before lung cancer diagnosis were defined as former smokers.
+All patients who received neoadjuvant and adjuvant chemotherapy received platinum (cisplatin or carboplatin), and the chemotherapy regimen most frequently administered was carboplatin-taxol combination.

KDR Copy Number Analysis in Tumor Specimens.

Two methodologies were utilized to test KDR CNG in NSCLC tumor specimens: real-time quantitative PCR (qPCR) and fluorescence in situ hybridization (FISH). To enrich for malignant cell content for qPCR analysis, tumor tissues were manually microdissected from optimal cutting temperature (OCT) compound-embedded frozen tissue sections for subsequent DNA extraction. Tumor DNA was extracted using Pico Pure DNA Extraction Kit (Arcturus, Mountain View, Calif.) according to the manufacturer's instructions. DNA samples with proportions of microdissected tumor cell greater than 70% were qualified for qPCR analysis. KDR gene copy number was detected by real-time quantitative PCR (qPCR) using the ABI 7300 real time PCR system (Applied Biosystems, Foster City, Calif.). The primers used to amplify KDR were KF-5′-GACACACCCTCAGGCTCTTG-3′ (SEQ ID NO:1) and KR-5′-ACTTTTCACCGCCTGTTCTC-3′ (SEQ ID NO:2). Each PCR was performed using Power SYBR Green PCR Master Mix (Applied Biosystems, Foster City, Calif.) at 50° C. for 2 min and 95° C. for 10 min followed by 40 cycles at 95° C. for 15 s and 60° C. for 1 min. β-Actin was introduced as the endogenous reference gene and TaqMan Control Human Genomic DNA (Applied Biosystems, Foster City, Calif.) was amplified as a standard control for calibration. All sample and standard DNA reactions were set in triplicate to gauge reaction accuracy. The target gene copy number was quantified using the comparative Cₜ method. Gene copy number of greater than 4 was considered as CNG, as previously reported.

KDR copy number analysis in NSCLC malignant tumor cells was also performed using a dual-color FISH assay. The KDR probe was prepared from the BAC clone RP11-21A18 obtained from CHORI (Oakland, Calif.). The following set of primers was used to confirm the inclusion of the sequences of interest by touchdown PCR: 5′-TGAGACTTGAGCAATCACTAGGCT-3′ (SEQ ID NO:3) and 5′-TAACCAAGGTACTTCGCAGGGATT-3′ (SEQ ID NO:4). DNA was purified from a single-cell colony (Qiagen QIAamp DNA Mini Kit) and amplified (Qiagen repli-G kit) per the manufacturer's instructions. DNA was labeled in 1 μg aliquots by nick translation (Vysis Nick Translation Kit, Des Plaines, Ill.) with SpectrumRed (SR) conjugated dUTPs, ethanol precipitated with herring sperm and human Cot-1, and the pellet resuspended in t-DenHyb (Insitus Biotechnologies, Albuquerque, N. Mex.). The KDR probe was validated in normal specimens for chromosomal mapping and appropriate specificity and sensitivity. A similarly constructed probe mapping to 6p21 (VEGFA) and labeled in SpectrumGreen was used as an internal control. The four-micron thick sections were incubated for two hours to overnight at 56° C., deparaffinized in Citri-Solv (Fisher, Waltham, Mass.), and washed in 100% ethanol. The slides were sequentially incubated in 2× saline-sodium citrate buffer (SSC) at 75° C. for 18-23 min, digested in 0.5 mg/mL proteinase K/2×SSC at 45° C. for 18-23 min, washed in 2×SSC for 5 min, and dehydrated in ethanol. Probe was applied to the selected hybridization area using 25-100 ng of KDR per 113 mm² area, which was covered with a glass coverslip and sealed with rubber cement. DNA denaturation was performed for 15 min at 85° C. and hybridization was allowed to occur at 37° C. for 36-48 hours. Post-hybridization washes were performed sequentially with 2×SSC/0.3% Nonidet P-40 (NP40) (pH 7.0-7.5) at 72° C. for 2 min and 2×SSC for 2 min, followed by dehydration in ethanol. Chromatin was counterstained with DAPI (0.3 μg/mL in Vectashield mounting medium, Vector Laboratories). Gene copy number analysis was done in approximately 50 nuclei per tumor in at least four areas, and the selection of the area was guided by images captured in the H&E-stained section. Greater than two gene copies per cell on average was considered as CNG.

KDR Copy Number and VEGFR-2 and HIF-1α Expression Analyses in Cell Lines.

Whole-genome SNP array profiling was performed in 75 NSCLC cell lines using the Illumina Human1M-Duo DNA Analysis BeadChip (Illumina, Inc., San Diego, Calif.). Prior to analysis, SNP data were normalized to the regional baseline copy number to account for aneuploidy. For VEGFR-2 reverse phase protein array (RPPA) analysis performed in 63 NSCLC cell lines, protein lysate was collected from sub-confluent cultures after 24 hours growth in media with 10% fetal bovine serum (FBS) and assayed by RPPA as previously described (Cheng et al., 2005; Byers et al., 2009). Cisplatin and carboplatin sensitivity was determined by MTS (3-(4,5-dimethylthiazol-2-yl)-5-(3-carboxymethoxyphenyl)-2-(4-sulfophenyl)-2H-tetrazolium, inner salt) assay for each cell line and the concentration required for 50% growth inhibition (IC₅₀) was determined. MTS assays were repeated at least three times for each cell line and the mean IC₅₀ value used for analysis. For HIF-1 expression analysis, the cells were serum-starved for 24 h and stimulated with 50 ng/mL VEGF-A (R&D Systems, MN, USA). Cells were incubated in normoxia and protein lysates were collected after 8 h. HIF-1α ELISA (R&D Systems, MN, USA) was performed according to the manufacturer's instructions.

Microvascular Density (MVD), VEGFR-2 and HIF-1α Expression Analyses in Tumors.

Histology sections were incubated at room temperature with primary antibodies against VEGFR-2 (dilution 1:50, Abcam, Cambridge, Mass.) for 90 min, CD34 (dilution 1:100, Lab Vision, Fremont, Calif.) for 35 min, and HIF-1α (dilution 1:100, Novus Biologicals, Littleton, Colo.) for 65 min. Tissue sections were then incubated with the secondary antibody (EnVision Dual Link+; DAKO, Carpinteria, Calif.) for 30 min, after which diaminobenzidine chromogen was applied for 5 min.

Protein expression was quantified by immunohistochemistry using light microscopy with a 200× magnification by two observers who were blinded to the clinical and other molecular variables. Tissue samples were analyzed for VEGFR-2 expression in the cytoplasm and membrane of malignant cells, and for HIF-1α in the nucleus. A 4-value intensity score (0, 1+, 2+, 3+) was used and the percentage (0% to 100%) of the extent of reactivity. The final score was obtained by multiplying the intensity and extent-of-reactivity values (range, 0 to 300). MVD was assessed by AriolR 2.0 Image System (AriolR, Genetix, San Jose, Calif.) using the criteria of Weidner et al. (1991). The areas of highest neovascularization were identified as the regions of invasive carcinoma with the highest numbers of discrete microvessels stained for CD34. Any brown-stained endothelial cell or endothelial cell cluster that was clearly separate from adjacent microvessels, tumor cells, and other connective tissue elements was considered a single, countable microvessel. As previously published (Weidner et al., 1991), the numbers of CD34-positive vessels were counted in three selected hotspots consisting of a 200× magnification field (0.6 mm² field area), and the MVD and vessel areas were defined as the mean count of microvessels or vessel area (mm²) per 0.6-mm² field area.

Small Interfering RNA (siRNA) Transfection, Platinum Cytotoxicity, and Cell Migration Assays in Cell Lines.

NSCLC cells were transfected with siRNA targeting KDR and control siRNA (OriGene Technology, Md., USA) at a final concentration of 10 nM using Lipofectamine RNAiMAX (Invitrogen, CA, USA) according to the manufacturer's instructions. Medium was replaced after 24 h. To verify the knockdown efficiency, mRNA and protein of transfected cells were collected for real-time RT-PCR and Western blot analyses.

The assessment of in vitro resistance to cisplatin and carboplatin was determined by the MTS assay. NSCLC cell lines were seeded in octuplicate at a density of 2,000 per well in 96-well plates. The following day, cells were treated with cisplatin and carboplatin at various concentrations ranging from 0 to 120 μmol/L for cisplatin and 0 to 200 μmol/L for carboplatin. After 72 h of drug exposure, 20 μL of MTS solution was added per well. Cells were incubated for 1-4 hours at 37° C. and read at a wavelength of 490 nm.

The cell migration assay using NSCLC cell lines was performed (Nilsson et al., 2010). A total of 700 mL of serum-free RPMI with or without VEGF-A (50 ng/mL) was added to the lower compartment of the 24-well transwell migration inserts (8.0 μm pore size; BD Biosciences, NJ, USA). Cells (5×10⁴) were added to the upper chambers and incubated for 24 h. Cells in the upper compartment were removed by mechanical scraping, and cells that migrated to the underside of the membrane were stained and counted in a light microscope using a 40× magnification, as previously described.

KDR Mutation and SNPs Genotyping Analyses.

For KDR mutation analysis in NSCLC cell lines, exons 7, 11, 21, 26, 27 and 30 were examined using PCR-based sequencing and intron-based PCR primers. Primer sequence for KDR mutation detection were as follows: Ex7F, 5′-TTTGGAAGTTCAGTCAACTC-3′ (SEQ ID NO:5), Ex7R, 5′-ATCTCACTTGTCAAGGCACAG-3′ (SEQ ID NO:6); Ex11F, 5′-TGCGCTGTTATCTCTTTCTT-3′ (SEQ ID NO:7), Ex11R, 5′-AATCTCCAATATGCCTCACA-3′ (SEQ ID NO:8); Ex21F, 5′-TTGATGTCCTCCTTGTCTGC-3′ (SEQ ID NO:9), Ex21R, 5′-CATGCAGGAAGCACTAGCC-3′ (SEQ ID NO:10); Ex26F, 5′-CAGCATTCAGGAAGAAAGAGG-3′ (SEQ ID NO:11); Ex26R, 5′-GCTTCTTGGATGGAGGTGAC-3′ (SEQ ID NO:12); Ex27F, 5′-AAGCCATAACAACAGTCTTCTGTG-3′ (SEQ ID NO:13), Ex27R, 5′-GAGATGGCCTTGAAGTCACC-3′ (SEQ ID NO:14); Ex30-1F, 5′-CTGCCAACTCCTTTGTTTGC-3′ (SEQ ID NO:15); Ex30-1R, 5′-CGGTTTGCACTCCAATCTCT-3′ (SEQ ID NO:16); Ex30-2F, 5′-AAGGCTCAAACCAGACAAGC-3′ (SEQ ID NO:17), Ex30-2R, 5′-TCATGTGATGTCCAGGAGTTG-3′ (SEQ ID NO:18).

Each PCR was done using HotStar Taq Master Mix (Qiagen, Valencia, Calif.) for 40 cycles at 94° C. for 30 s, 59° C. for 30 s, and 72° C. for 30 s, followed by a 7-min extension at 72° C. Mutation and SNP genotyping were performed using the ABI Prism 7900 Sequence Detection System (Applied Biosystems, Foster City, Calif.). SNP genotyping was performed by laboratory personnel blinded to patient status, and all procedures were repeated on a randomly selected 5% of the samples in order to validate the genotyping accuracy.

Statistical Analysis.

Demographic and clinical information were compared using the Chi-square or Fisher exact tests for category variables, and Wilcoxon rank-sum or Kruskal-Wallis tests for continuous variables. The distributions of overall survival (OS) and recurrence-free survival (RFS) were estimated by the Kaplan-Meier method and compared between groups using the log-rank test. Cox proportional hazard models were used for regression analyses of survival data and conducted on OS defined as time from surgery to death or last contact, and on RFS defined as time from surgery to recurrence or last contact. Follow-up time was censored at 5 years. For the correlation analysis of KDR CNG in NSCLC cell lines using the whole-genome SNP arrays data with cisplatin sensitivity, the Wilcoxon rank sum test was used. The NSCLC cell lines RPPA data was quantified using the SuperCurve method, which detects changes in protein level as previously reported.

Results

KDR Gene CNG Analysis.

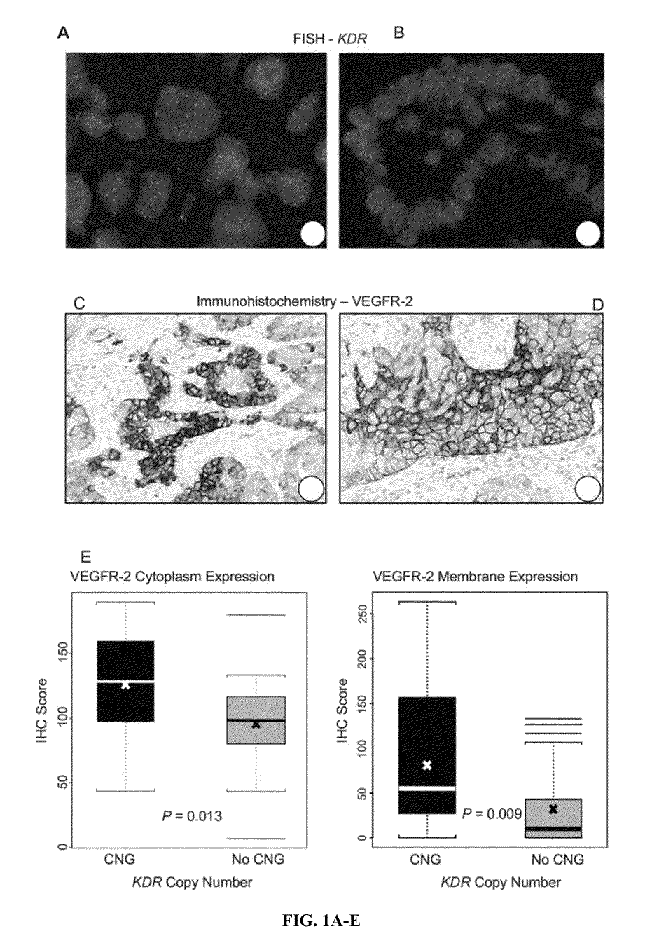

In epithelial malignant NSCLC cells microdissected from tumor tissues, KDR CNG was detected in 45 (32%) of 139 tumors examined. Similar frequency of KDR CNG was found in adenocarcinoma (26/85, 31%) and squamous cell carcinoma (19/54, 35%) histologies (P=0.572). The range of increased KDR copy numbers was from 4 to 11 gene copies. None of 15 normal tissue samples adjacent to the NSCLC tested showed KDR CNG. To confirm KDR CNG results by qPCR, 20 tumor specimens with KDR CNG by qPCR were examined by FISH. KDR copy gains in the malignant cells were confirmed by FISH in all 20 NSCLC specimens detected by qPCR (FIG. 1A).

Correlation Between KDR CNG and VEGFR-2 Protein Expression and MVD.

To assess the immunohistochemical expression of VEGFR-2 in NSCLC malignant cells and the MVD (CD34) in lung tumor tissue stroma, 52 lung tumor specimens with whole histologic sections from FFPE tissues were selected. Of these, 26 cases had KDR CNG and 26 cases did not. VEGFR-2 protein expression was present both in the cytoplasm and membrane of malignant cells as well as in vessel endothelial cells (FIG. 1B).

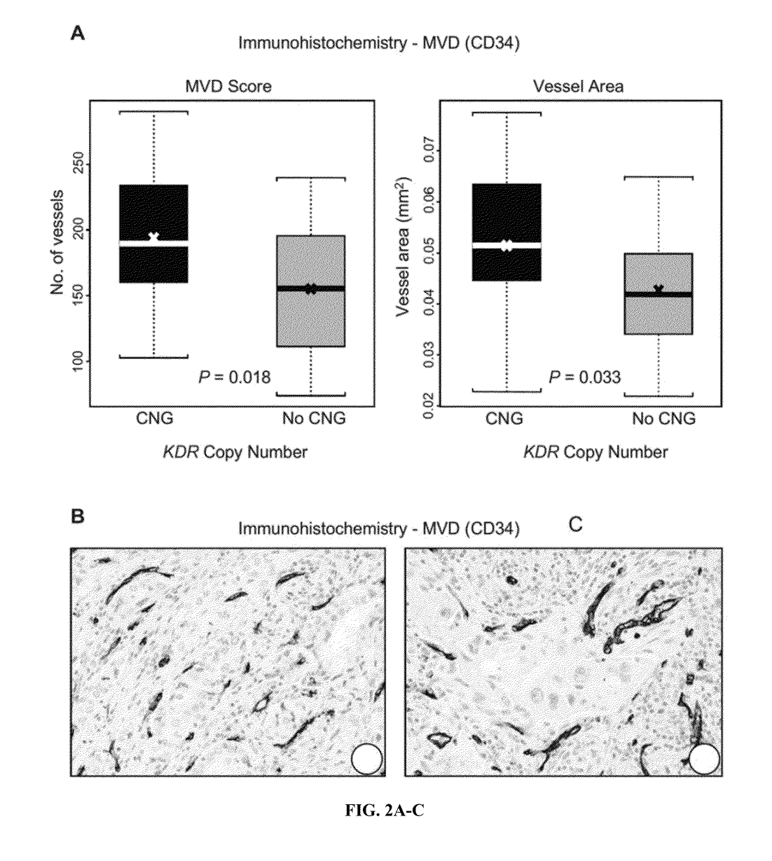

Levels of VEGFR-2 expression in cytoplasm and in membrane were associated with KDR CNG in malignant cells of NSCLC. Tumors with KDR CNG showed significantly higher cytoplasmic (P=0.013) and membrane (P=0.009) VEGFR-2 protein expression in the malignant cells (FIG. 1C), and higher MVD (P=0.018) and larger vessel areas (P=0.033) in the tumor stroma than cases without KDR CNG (FIGS. 2A and 2B).

Association Between Tumor KDR CNG, Clinicopathologic Features, and Clinical Outcome.

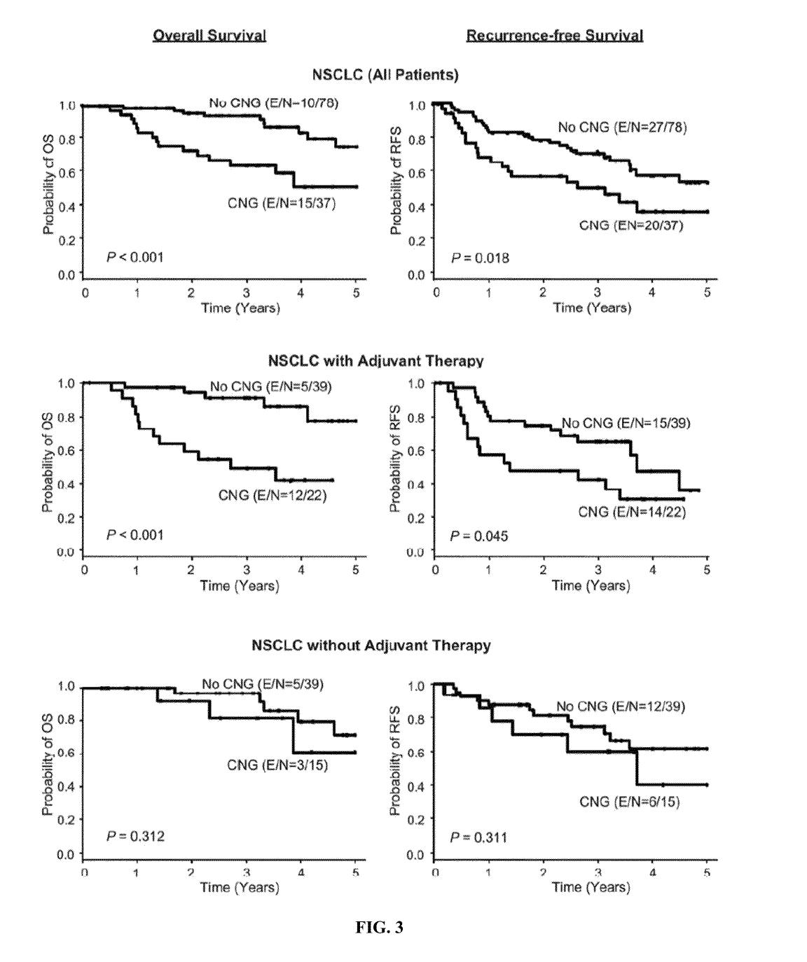

When KDR CNG was correlated with patients' clinicopathologic features, no correlation with tumor histology, smoking status, and tumor stage was found. Interestingly, in the multivariate analysis after adjusting for stage and adjuvant therapy, KDR CNG was associated with poor OS (HR=4.0; 95% CI, 1.76 to 9.07; P=0.001) and shortened RFS (HR=1.83, 95% CI, 1.02 to 3.29; P=0.044) in 115 NSCLC patients who underwent surgical resection. Strikingly, KDR CNG was associated with a significantly worse OS (HR=5.16, 95% CI, 1.75 to 15.2, P=0.003) in NSCLC patients receiving platinum adjuvant therapy, but not in patients without adjuvant therapy (P=0.349) (FIG. 3 and Table 2).

TABLE 2
Multivariate analysis for outcome by KDR copy gain in non-small cell
lung carcinoma (NSCLC) patients by adjuvant chemotherapy.

| | | | | Adjusted | |
|---|---|---|---|---|---|
| | | | | Hazard Ratio | |
| Cases | N | Comparison | Outcome | (HR)* (95% CI) | P |
| All | 115 | Gain vs. no | OS^{±} | 4.00 (1.76, 9.07) | 0.001 |
| patients | | gain | | | |
| | | | RFS⁺ | 1.83 (1.02, 3.29) | 0.044 |
| Adjuvant | 61 | Gain vs. no | OS | 5.16 (1.75, 15.2) | 0.003 |
| therapy | | gain | | | |
| | | | RFS | 1.87 (0.9, 3.92) | 0.1 |
| No | 54 | Gain vs. no | OS | 1.99 (0.47, 8.4) | 0.349 |
| adjuvant | | gain | | | |
| therapy | | | | | |
| | | | RFT | 1.83 (0.66, 5.05) | 0.243 |

These data suggest that KDR CNG in malignant cells may represent a predictive marker of worse outcome in patients with surgically resected NSCLC treated with platinum-based adjuvant chemotherapy.

The impact of neoadjuvant chemotherapy on KDR CNGs was also examined. The platinum neoadjuvant-treated tumors (33%, 8/24) had similar frequency of KDR CNGs than cases without neoadjuvant therapy (32%, 37/115).

KDR CNG and VEGFR-2 Protein Levels and Correlation with Platinum Resistance in Cell Lines.

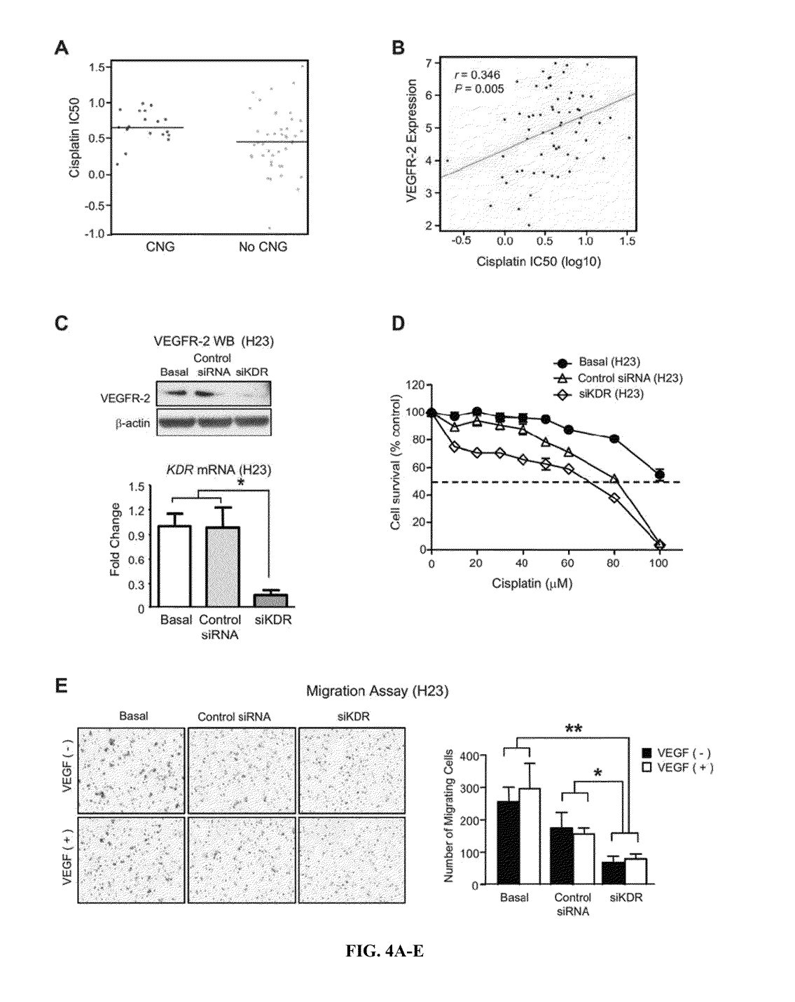

The association detected between KDR CNG and worse outcome in patients treated with platinum adjuvant therapy prompted us to examine the correlation between KDR gain and VEGFR-2 protein levels in NSCLC cell lines with in vitro resistance to platinum drugs. KDR CNG was assessed by SNP array analysis in 75 NSCLC cell lines. Cell lines with KDR copy gains of 6-9 copies or ≧10 copies above the regional baseline copy number were identified. Nineteen (25%) cell lines showed KDR CNG defined as ≧6 copies. Of these, three (4%) cell lines contained high-level gains (≧10 copies), and 16 (21%) had CNG where gene copy number was between 6 and 9. Of interest, cisplatin sensitivity in cell lines with ≧6 KDR copies demonstrated significantly more resistance to cisplatin (P=0.0179) (FIG. 4A).

Then, the expression of VEGFR-2 protein in a panel of 63 untreated NSCLC cell lines was correlated by RPPA with each cell line's sensitivity to cisplatin or carboplatin. Higher VEGFR-2 expression levels were significantly associated with resistance to both cisplatin (FIG. 4B) and carboplatin by Pearson correlation. The correlation coefficient (r) between VEGFR-2 expression and the concentration of cisplatin and carboplatin required to inhibit cell growth by 50% (IC₅₀) were 0.346 (P=0.005) and 0.319 (P=0.011), respectively.

Effect of KDR Knockdown on Platinum Sensitivity and Cell Migration in Cell Lines.

To investigate the role of KDR CNG and VEGFR-2 overexpression in resistance to both cisplatin and carboplatin, siRNA was utilized to knockdown KDR expression in H23 and H461 NSCLC cell lines, which contain 6-9 KDR gene copies. In both cell lines, siRNA targeting KDR significantly decreased KDR mRNA expression by real-time RT-PCR, and VEGFR-2 expression by Western blot, compared with control cells transfected with scrambled siRNA and nontransfected cells (P<0.05; FIG. 4C). To evaluate the effect of KDR overexpression on sensitivity to cisplatin and carboplatin, the expression of KDR was inhibited by transfecting H23 and H461 cells with control siRNA or siRNA targeting KDR and then treating the cells with increasing concentrations of the chemotherapy drugs. Cell viability was evaluated by MTS assay. The sensitivity of H23 cells to cisplatin (FIG. 4D) or carboplatin treatment was increased in siKDR transfected cells compared with control siRNA-transfected or untransfected cells, suggesting that VEGFR-2 is contributing to chemoresistance in this model.

Whether VEGFR-2 could promote tumor cell migration was investigated next. Using the Boyden chamber assay, we observed that knockdown or reduction of VEGFR-2 expression induced by siKDR transfection significantly inhibited the migration of H23 cells compared with siRNA control-transfected or untransfected cells (FIG. 4E). Cells with KDR CNGs were also more sensitive to inhibition with drugs targeting KDR, PDGFR, and KIT, such as sunitinib.

Correlation Between KDR CNG and HIF-1α Expression in Cell Lines and Tumors.

The observations that KDR CNGs were associated with increased angiogenesis, chemoresistance, and migration suggested that VEGFR-2 may be impacting the HIF-1α pathway, which is known to impact each of these cellular properties (Nilsson et al., 2010; Roybal et al., 2010).

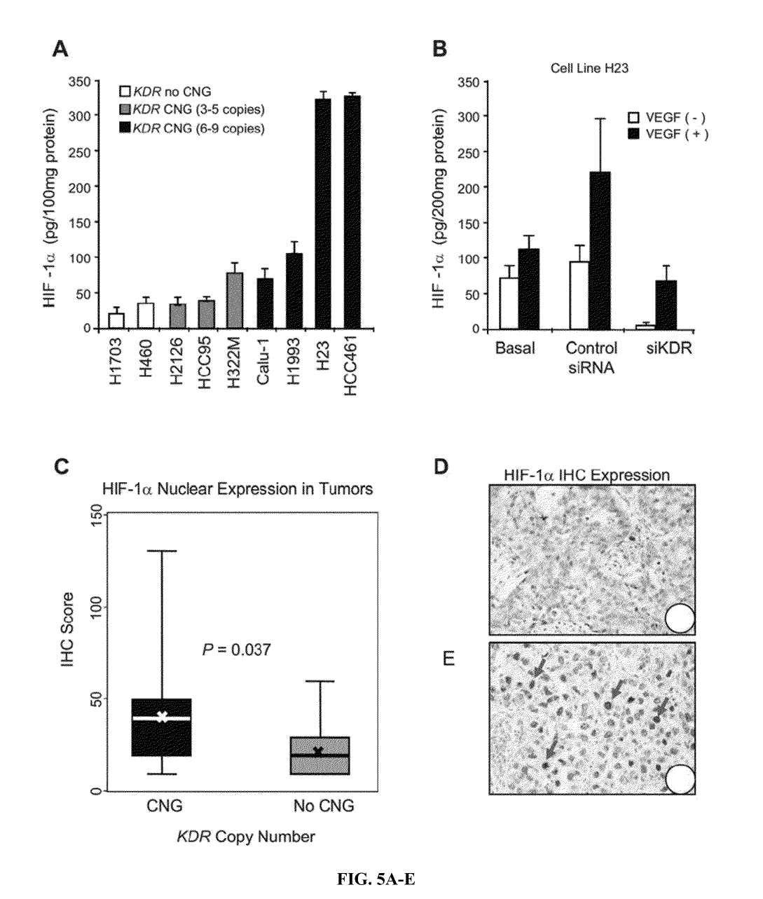

To investigate this further, HIF-1α levels were evaluated by ELISA in a panel of NSCLC cell lines with a range of KDR copy numbers and expression of VEGFR-2. HIF-1α levels were higher in cell lines with KDR CNG, and significantly (P=0.02) higher in cells with 6-9 gene copies, compared to cells with no CNG (FIG. 5A). In H23 cells, which have KDR CNG, stimulation with 50 ng/mL VEGF-A for 8 h induced a rise in HIF-1α expression. Furthermore, knockdown of KDR with siRNA significantly (P=0.01) reduced HIF-1α levels (FIG. 5B). These data indicated that VEGFR-2 can regulate HIF-1α in a ligand-dependent, but hypoxia-independent, manner in NSCLC cells.

The association between KDR CNG and HIF-1α in NSCLC clinical specimens was investigated next. Similarly to the results in the NSCLC cell lines, tumor tissue specimens with KDR CNG (n=25) demonstrated a significantly (P=0.037) higher expression of nuclear HIF-1α expression by immunohistochemistry than tumors without CNG (n=22) (FIGS. 5C and 5D).

KDR Mutation and SNP Analyses.

To investigate whether alterations in the KDR gene other than CNGs may impact NSCLC tumors, the inventors assessed the KDR gene for mutations and SNPs. For KDR mutation analysis in NSCLC cell lines, the inventors examined 6 KDR exons shown to be mutant in adenocarcinoma tumors (Ding et al., 2008; Bernatchez et al., 1999; Carrillo de Santa Pau et al., 2009; Weidner et al., 1991; Koukourakis et al., 2002; Tan et al., 2009; Qi et al., 2001). In 37 tested NSCLC cell lines, only two mutations in the KDR gene were found, an intronic T+2A exon 11 mutation in HCC2450 and a CGT946CAT point mutation in exon 21 in HCC2279. No mutation affecting exons 11 or 21 was detected in 200 NSCLC tissues specimens examined.

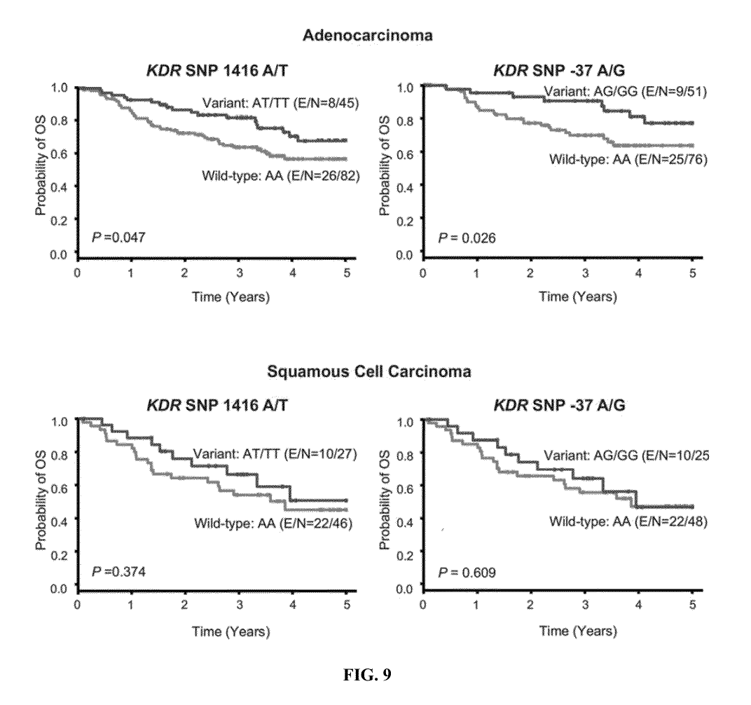

In addition, three KDR SNPs (889G/A, 1416A/T, and −37A/G) were genotyped in DNA extracted from 200 NSCLC tumors and correlated with patients clinicopathologic features, including outcome (Table 3). No correlation was found between the SNP genotypes distribution and OS or RFS of all NSCLC patients examined. When the data were analyzed by tumor histology, among the 127 lung adenocarcinoma patients examined, both KDR 1416 AT/TT (HR=0.45; 95% CI, 0.2 to 0.99; P=0.048) and −37 AG/GG (HR=0.43; 95% CI, 0.2 to 0.92; P=0.031) variant genotypes were associated with a favorable OS in the multivariate analysis after adjusting for tumor stage and neoadjuvant therapy (FIG. 9 and Table 4). However, no KDR SNP genotype was associated with OS in lung squamous cell carcinoma patients (FIG. 9). Moreover, no genotype in the three KDR SNPs was associated with RFS in NSCLC patients divided by histology type.

TABLE 3
Distribution of genotypes in three KDR single nucleotide
polymorphisms (SNP) in non-small cell lung carcinoma (NSCLC).

| KDR SNP | ID in NCBI^{±} | Genotype | Type | Case (%) |
|---|---|---|---|---|
| 889 | rs2305948 | GG | Wild type | 155 (78) |
| | | GA | Variant type | 41 (20) |
| | | AA | Variant type | 4 (2) |
| 1416 | rs1870377 | AA | Wild type | 128 (64) |
| | | AT | Variant type | 63 (32) |
| | | TT | Variant type | 9 (4) |
| −37 | rs2219471 | AA | Wild type | 124 (62) |
| | | AG | Variant type | 68 (34) |
| | | GG | Variant type | 8 (4) |

^{±}NCBI, National Center for Biotechnology Information.

TABLE 4
Multivariate analysis for overall survival in three KDR single nucleotide
polymorphisms (SNP) in non-small cell lung carcinoma (NSCLC).

| | | | Adjusted Hazard | |
|---|---|---|---|---|
| | KDR | | Ration (HR)* | |
| Cases | SNP | Genotype | (95% CI) | P |
| NSCLC | 889 | GA/VA vs GG | 0.92 (0.51 to 1.66) | 0.78 |
| | 1416 | AT/TT vs. AA | 0.59 (0.34 to 1.01) | 0.056 |
| | −37 | AG/GG vs. AA | 0.6 (0.35 to 1.03) | 0.62 |
| Adenocarcinoma | 889 | GA/AA vs. GG | 0.63 (0.24 to 1.65) | 0.348 |
| | 1416 | AT/TT vs. AA | 0.45 (0.2 to 0.99) | 0.048 |
| | −37 | AG/GG vs. AA | 0.43 (0.2 to 0.92) | 0.031 |
| Squamouns cell | 889 | GA/AA vs. GG | 1.16 (0.53 to 2.51) | 0.713 |
| carcinoma | | | | |
| | 1416 | AT/TT vs. AA | 0.76 (0.36 to 1.61) | 0.468 |
| | −37 | AG/GG vs. AA | 0.84 (0.4 to 1.78) | 0.649 |

*Adjusting for tumor stage; follow-up is censored at 5 years.

Furthermore, among NSCLC patients with the KDR 889 GA/AA variant genotypes, those who received platinum neoadjuvant and/or adjuvant chemotherapy showed a significantly better OS (HR=0.22; 95% CI, 0.05 to 0.94; P=0.041) than patients who did not receive chemotherapy in the multivariate analysis after adjusting for histology and tumor stage. However, no survival benefit was found in NSCLC patients with KDR 889 GG wild genotype (HR=1.23; 95% CI, 0.64 to 2.35; P=0.538).

Finally, all KDR SNP genotypes were compared with primary tumor expression for VEGFR-2 and MVD in 52 NSCLC specimens. However, no genotypes correlated with the expression of any of these markers in NSCLC tumors.

### Example II

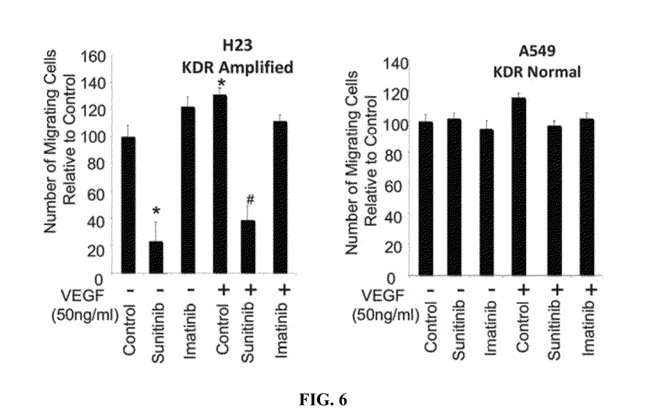

The inventors observed that in KDR amplified cell lines, inhibition of the VEGFR pathway using the multitargeting TKI sunitinib (which has activity against VEGFR, PDGFR, and Kit) results in a decrease in cellular migration. However, imatinib, which targets BCL/ABL, Kit, and PDGFR, does not inhibit cellular migration, suggesting a role for VEGFR in migration. In contrast, the VEGFR inhibitor, sunitinib, has no effect on migration of A549 cells which do not have amplification of VEGFR. Representative data are shown in FIG. 6.

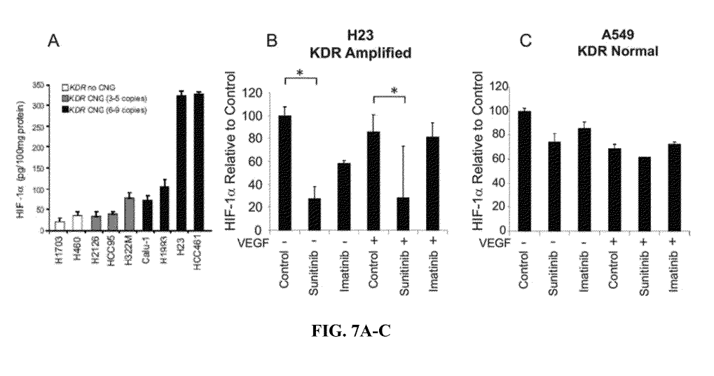

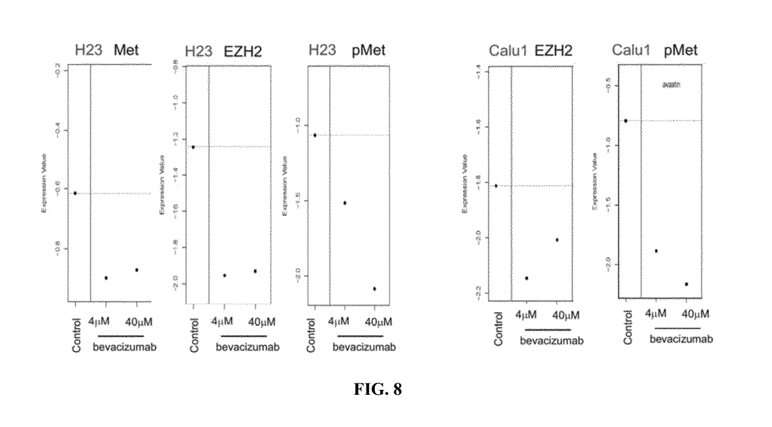

In lung cancer as well as in neuroblastoma cells, multiple receptor tyrosine kinases, including VEGFR1, EGFR, PDGFR, and RET, can drive HIF-1α levels. Therefore, whether VEGFR drives HIF-1α expression in NSCLC cells with VEGFR amplification was investigated. Higher levels of HIF-1α were observed in cell lines with VEGFR CNGs compared to those without (FIG. 7A). H23 cells (KDR CNG+) were treated with the VEGFR inhibitor sunitinib and a statistically significant decrease in HIF-1α levels was observed as determined by ELISA assay (FIG. 7B). Imatinib, which does not inhibit VEGFR, did not affect HIF-1α levels. No change in HIF-1α levels were detected in A549 cells, which do not contain VEGF CNGs (FIG. 7C). In addition, two VEGFR amplified cell lines, H23 and Calu1, were treated with the VEGFR pathway inhibitor bevacizumab and changes in proteins regulated by HIF-1α were evaluated. As shown in FIG. 8, multiple HIF-1α-regulated proteins were decreased in the presence of bevacizumab, including EZH2, Met, and phosphorylated Met.

### Example III

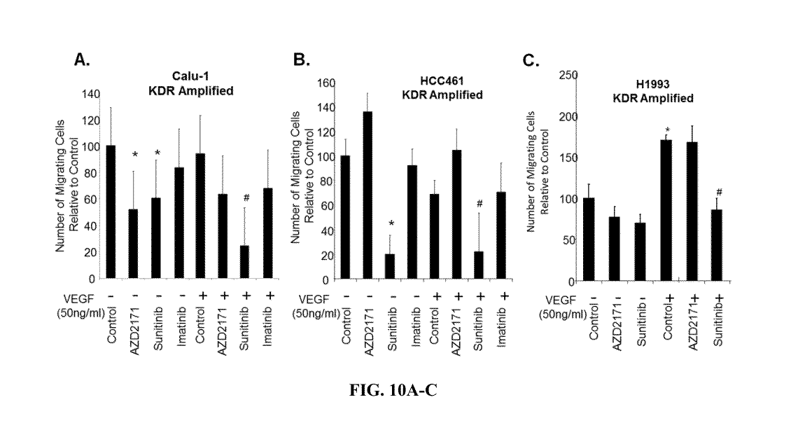

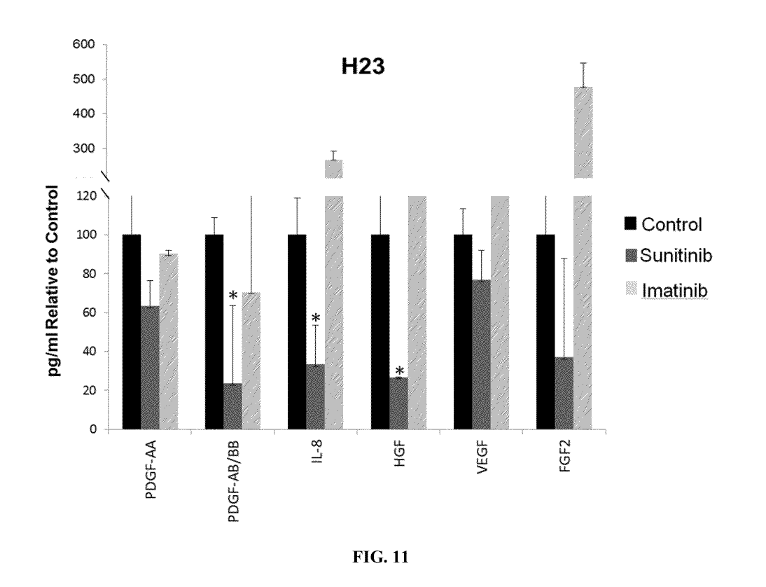

The inventors further evaluated the effect of VEGF and VEGFR TKIs on tumor cell migration using additional NSCLC cell lines with KDR CNGs (Calu1, HCC461, and H1993). Similar to the previous observations, VEGFR TKIs decreased tumor cell migration (FIG. 10). Because the inventors found VEGFR TKIs to decrease HIF-1α levels in NSCLC cells with KDR CNGs, and HIF-1α is a key regulator of many angiogenic factors, the inventors next investigated the effect of VEGFR TKIs on tumor cell secretion of cytokines including VEGF, PDGF, IL-8, HGF, and FGF2. H23 tumor cells were treated with control media or media containing the VEGFR TKI sunitinib (1 μM) for 24 hours. Conditioned media was collected and cytokine levels were assessed by ELISA assay. VEGFR inhibition resulted in significantly decreased levels of tumor-derived PDGF-AB/BB, IL-8, and HGF (FIG. 11). Imatinib was used as a negative control as it does not inhibit VEGFR.

### Example IV

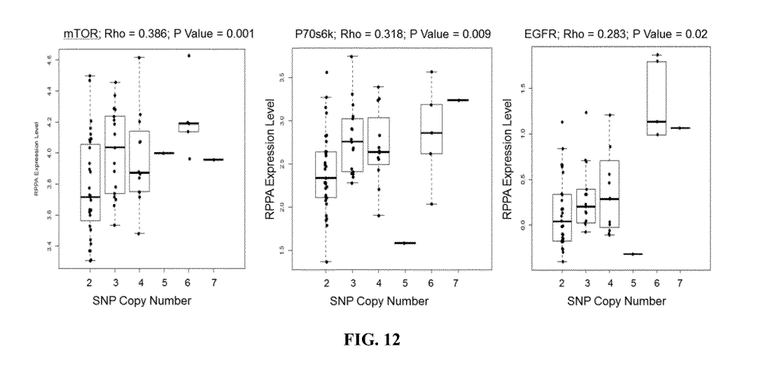

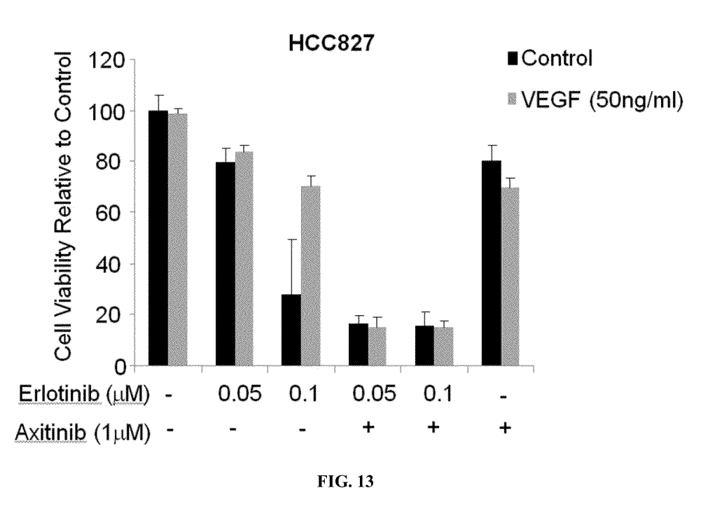

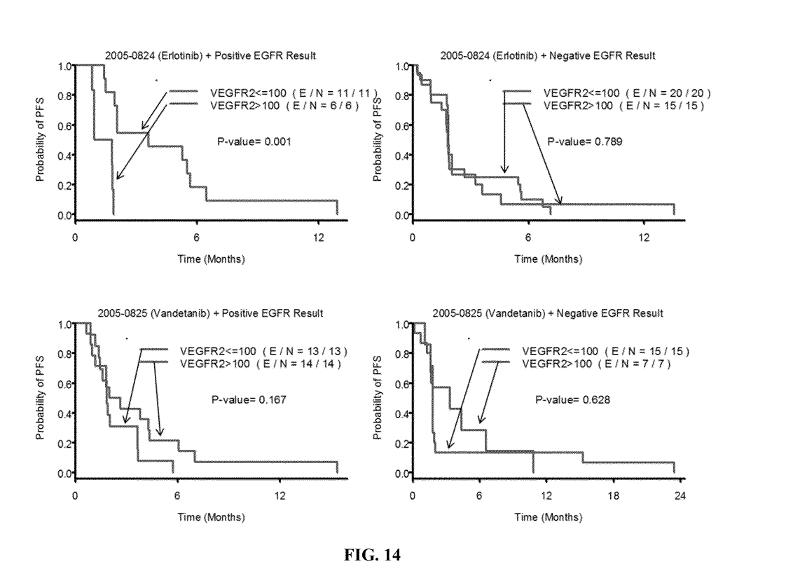

To investigate signaling pathways that may be differentially expressed between tumor cells with or without KDR CNGs, the inventors compared KDR copy number with expression of a broad panel of proteins screened by reverse phase protein array (RPPA). Cell lines with high copy numbers of KDR had significantly greater expression of mTOR pathway components (mTOR and p70s6K). In addition, KDR CNG was associated with increased levels of EGFR (FIG. 12). The inventors next evaluated whether VEGFR might promote erlotinib resistance. The inventors treated HCC827 cells, which harbor the EGFR activating mutation, with VEGF with or without the VEGFR TKI axitinib. After 24 hours, increasing concentrations of erlotinib were added to the cells. VEGF increased tumor cell survival in the presence of erloninib, whereas axitinib reversed the effect (FIG. 13). Furthermore, in clinical specimens from the BATTLE clinical trial, patients who had EGFR-driven disease and were treated with erlotinib did worse when they had high levels of VEGFR2, in comparison with those with low levels of VEGFR2 (P=0.001; FIG. 14).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Ballas et al., Rationale for targeting VEGF, FGF, and PDGF for the treatment of NSCLC, Onco. Targets Ther., 4:43-58, 2011.
Bernatchez et al., Vascular Endothelial Growth Factor Effect On Endothelial Cell Proliferation, Migration, and Platelet-Activating Factor Synthesis is Flk-1-Dependent, J. Biol. Chem., 274:31047-31054, 1999.
Byers et al., Reciprocal Regulation of c-Src and STAT3 in Non-Small Cell Lung Cancer, Clin. Cancer Res., 15:6852-6861, 2009.
Carrillo de Santa Pau et al., Prognostic Significance of the Expression of Vascular Endothelial Growth Factors A, B, C, and D and their Receptors R1, R2 and R3 in Patients with Nonsmall Cell Lung, Cancer, 115:1701-1712, 2009.
Cheng et al., Assay of Rab25 Function in Ovarian and Breast Cancers, Methods Enzymol., 403:202-215, 2005.
Ding et al., Somatic Mutations Affect Key Pathways in Lung Adenocarcinoma, Nature, 455:1069-1075, 2008.
Donnem et al., Inverse Prognostic Impact of Angiogenic Marker Expression in Tumor Cells Versus Stromal Cells in Non Small Cell Lung Cancer, Clin. Cancer Res., 13:6649-6657, 2009.
Engelman et al., MET amplification leads to gefitinib resistance in lung cancer by activating ERBB3 signaling, Science, 316:1039-1043, 2007.
Ferrara et al., The Biology of Vascular Endothelial Growth Factor, Endocr. Rev., 18:4-25, 1997.
Fidler et al., The Implications of Angiogenesis for the Biology and Therapy of Cancer Metastasis, Cell, 79:185-188, 1994.
Folkman, Tumor Angiogenesis: Therapeutic Implications, N. Engl. J. Med., 285:1182-1186, 1971.
Forsti et al., Polymorphisms in the KDR and POSTN Genes: Association with Breast Cancer Susceptibility and Prognosis, Breast Cancer Res. Treat, 101:83-93, 2007.
Hanahan et al., Hallmarks of Cancer: the Next Generation, Cell, 144:646-674, 2011.
Innis et al., PCR Protocols, A Guide to Methods and Applications, Academic Press, Inc. N.Y., 1990.
Hirami et al., Relation of Epidermal Growth Factor Receptor, Phosphorylated-Akt, and Hypoxia-Inducible Factor-1alpha in Non-Small Cell Lung Cancers, Cancer Lett., 214:157-164, 2004.
Ishii et al., Enhancement of Pleural Dissemination and Lymph Node Metastasis of Intrathoracic Lung Cancer Cells by Vascular Endothelial Growth Factors (VEGFs), Lung Cancer, 45:325-337, 2004.
Kijima et al., Regulation of cellular proliferation, cytoskeletal function, and signal transduction through CXCR4 and c-Kit in small cell lung cancer cells, Cancer Res., 62:6304-6311, 2002.
Koukourakis et al., Hypoxia-Inducible Factor (HIF1A and HIF2A), Angiogenesis, and Chemoradiotherapy Outcome of Squamous Cell Head-And-Neck Cancer, Int. J. Radiat. Oncol. Biol. Phys., 53:1192-1202, 2002.
Ludovini et al., Vascular Endothelial Growth Factor, P53, Rb, Bc1-2 Expression and Response To Chemotherapy In Advanced Non-Small Cell Lung Cancer, Lung Cancer, 46:77-85, 2004.
Lynch et al., Activating mutations in the epidermal growth factor receptor underlying responsiveness of non-small-cell lung cancer to gefitinib, N. Engl. J. Med., 350:2129-2139, 2004.
Mi et al., RNA Aptamer-Targeted Inhibition of NF-kappa B Suppresses Non-Small Cell Lung Cancer Resistance to Doxorubicin, Mol. Ther., 16:66-73, 2008.
Mok et al., Gefitinib or carboplatin-paclitaxel in pulmonary adenocarcinomas, N. Engl. J. Med., 361:947-957, 2009.
Mountain, Revisions in the International System for Staging Lung Cancer, Chest, 111:1710-1717, 1997.
Nilsson et al., Multiple Receptor Tyrosine Kinases Regulate HIF-1alpha and HIF-2alpha in Normoxia and Hypoxia in Neuroblastoma: Implications for AntiAngiogenic Mechanisms of Multikinase Inhibitors, Oncogene, 29:2938-2949, 2010.
Paez et al., EGFR mutations in lung cancer: correlation with clinical response to gefitinib therapy, Science, 304:1497-1500, 2004.
Phillips et al., Epidermal Growth Factor and Hypoxia-Induced Expression of CXC Chemokine Receptor 4 on Non-Small Cell Lung Cancer Cells is Regulated by The Phosphatidylinositol 3-Kinase/PTEN/AKT/Mammalian Target of Rapamycin Signaling Pathway and Activation of Hypoxia Inducible Factor-1alpha, J. Biol. Chem., 280:22473-22481, 2005.
Qi et al., VEGF-Induced Activation of Phosphoinositide 3-Kinase is Dependent on Focal Adhesion Kinase, Exp. Cell Res., 263:173-182, 2001.
Roybal et al., miR-200 Inhibits Lung Adenocarcinoma Cell Invasion and Metastasis by Targeting Flt1/VEGFR1, Mol. Cancer. Res., 9:25-35, 2010.
Seto et al., Prognostic Value of Expression of Vascular Endothelial Growth Factor and Its Flt-1 and KDR Receptors In Stage I Non-Small-Cell Lung Cancer, Lung Cancer, 53:91-96, 2006.
Tan et al., The Key Hypoxia Regulated Gene CAIX is Upregulated In Basal-Like Breast Tumours and is Associated with Resistance to Chemotherapy, Br. J. Cancer, 100:405-411, 2009.
Tao et al., Kinase Insert Domain Receptor (KDR) Extracellular Immunoglobulin-Like Domains 4-7 Contain Structural Features that Block Receptor Dimerization and Vascular Endothelial Growth Factor-Induced Signaling, J. Biol. Chem., 276:21916-21923, 2001.
Terman et al., Identification of a New Endothelial Cell Growth Factor Receptor Tyrosine Kinase, Oncogene, 6:1677-1683, 1991.
Waltenberger et al., Different Signal Transduction Properties of KDR and Flt1, Two Receptors for Vascular Endothelial Growth Factor, J. Biol. Chem., 269:26988-26995, 1994.
Weidner et al., Tumor Angiogenesis and Metastasis—Correlation in Invasive Breast Carcinoma, N. Engl. J. Med., 324:1-8, 1991.
Wen et al., Suppression of Cyclin D1 by Hypoxia-Inducible Factor-1 Via Direct Mechanism Inhibits the Proliferation and 5-fluorouracil-induced Apoptosis of A549 Cells, Cancer Res., 70:2010-2019, 2010.
Xu et al., Epidermal Growth Factor Receptor Regulates MET Levels and Invasiveness Through Hypoxia-Inducible Factor-1alpha In Non-Small Cell Lung Cancer Cells, Oncogene, 29:2616-2627, 2010.

1. A method of treating a cancer patient comprising:
(a) selecting a patient determined to have a cancer with an elevated KDR, PDGFR, or KIT level; and
(b) treating the patient with a VEGF/VEGFR, PDGFR, or KIT pathway inhibitor.

2. The method of claim 1, wherein the elevated KDR, PDGFR, or KIT level is further defined as a gain in gene copy number, increased mRNA expression, or increased protein expression.

3. The method of claim 1, wherein the elevated KDR level is further defined as an increased mRNA or protein level of a KDR-regulated gene.

4. The method of claim 3, wherein the KDR-regulated gene is HIF-1α.

5. The method of claim 1, wherein the cancer patient has a NSCLC or glioblastoma.

6. The method of claim 1, wherein the cancer is metastatic.

7. The method of claim 1, wherein the patient is treated with a VEGF/VEGFR pathway inhibitor.

8. The method of claim 1, wherein the patient is treated with a combination of two or more VEGF/VEGFR, PDGFR, or KIT pathway inhibitors.

9. The method of claim 1, further comprising treating the patient with a second anti-cancer therapy.

10. The method of claim 9, wherein the second anti-cancer therapy is not a platinum-based chemotherapeutic agent or an EGFR inhibitor.

11. The method of claim 1, wherein the patient has undergone surgery or radiotherapy and the treatment is an adjuvant treatment.

12. The method of claim 1, wherein the VEGF/VEGFR pathway inhibitor is ramucirumab, sunitinib, bevacizumab, aflibercept, BIBF1120, sorafenib, cediranib, dovitinib, pazopanib, ponatinib, semaxanib, axitinib, PP-121, telatinib, TSU-68. Ki8751, tivozanib, motesanib, regorafenib, vatalanib, or vandetanib.

13. The method of claim 1, wherein the PDGFR pathway inhibitor is imatinib, sunitinib, axitinib, BIBF1120, pazopanib, pnoatinib, MK-2461, dovitinib, crenolanib, PP-121, telatinib, CP 673451, TSU-68, Ki8751, tivozanib, masitinib, motesanib, MEDI-575, or regorafenib.

14. The method of claim 1, wherein the KIT pathway inhibitor is imatinib, axitinib, pazopanib, dovitinib, telatinib, Ki8751, tivozanib, masitinib, motesanib, sunitinib, 3G3, nilotinib, dasatinib, regorafenib, or vatalanib.

15. A method of predicting sensitivity of a cancer in a patient to VEGF/VEGFR, PDGFR, or KIT pathway inhibitors comprising:
(a) obtaining a sample of the cancer; and
(b) determining the KDR, PDGFR, and KIT level in the sample, wherein if the KDR, PDGFR, or KIT level is elevated, then the cancer is predicted to be sensitive to VEGF/VEGFR, PDGFR, or KIT pathway inhibitors.

16-28. (canceled)

29. A method of predicting sensitivity of a cancer in a patient to EGFR inhibitors or platinum-based chemotherapy comprising:
(a) obtaining a sample of the cancer; and
(b) determining the KDR level in the sample, wherein if the KDR level is not elevated, then the cancer is predicted to be sensitive to EGFR inhibitors or platinum-based chemotherapy.

30-39. (canceled)

40. A method of treating a cancer patient comprising:
(a) determining if the patient has a cancer that is sensitive to VEGF/VEGFR, PDGFR, or KIT pathway inhibitors according to claim 15; and
(b) treating the patient determined to have a cancer that is sensitive to VEGF/VEGFR, PDGFR, or KIT pathway inhibitors with VEGF/VEGFR, PDGFR, or KIT pathways inhibitors.

41-42. (canceled)

43. A method of determining a prognosis of a cancer patient comprising:
(a) obtaining a sample of the patient's cancer; and
(b) detecting polymorphisms at nucleotides−37 and 1416 in the KDR gene in the cells comprising the sample, wherein the cancer is determined to have a better prognosis if the −37 AG/GG and (or?) 1416 AT/TT polymorphisms are present.

44. A method of treating a cancer patient comprising:
(a) determining the cancer patient's prognosis according to claim 43; and
(b) applying an aggressive anticancer therapy if the polymorphisms are absent.

45. (canceled)

46. The method of claim 1 further comprising the step of determining the expression levels of VEGFR-2 in the biological sample wherein the presence of VEGFR-2 is further predictive of poor treatment outcome.

47-48. (canceled)


SEQUENCE LISTING (18 sequences)

SEQ ID NO: 1 - DNA, 20 bp, Artificial Sequence
  Synthetic Primer
gacacaccctcaggctcttg

SEQ ID NO: 2 - DNA, 20 bp, Artificial Sequence
  Synthetic Primer
acttttcaccgcctgttctc

SEQ ID NO: 3 - DNA, 24 bp, Artificial Sequence
  Synthetic Primer
tgagacttgagcaatcactaggct

SEQ ID NO: 4 - DNA, 24 bp, Artificial Sequence
  Synthetic Primer
taaccaaggtacttcgcagggatt

SEQ ID NO: 5 - DNA, 20 bp, Artificial Sequence
  Synthetic Primer
tttggaagttcagtcaactc

SEQ ID NO: 6 - DNA, 21 bp, Artificial Sequence
  Synthetic Primer
atctcacttgtcaaggcacag

SEQ ID NO: 7 - DNA, 20 bp, Artificial Sequence
  Synthetic Primer
tgcgctgttatctctttctt

SEQ ID NO: 8 - DNA, 20 bp, Artificial Sequence
  Synthetic Primer
aatctccaatatgcctcaca

SEQ ID NO: 9 - DNA, 20 bp, Artificial Sequence
  Synthetic Primer
ttgatgtcctccttgtctgc

SEQ ID NO: 10 - DNA, 19 bp, Artificial Sequence
  Synthetic Primer
catgcaggaagcactagcc

SEQ ID NO: 11 - DNA, 21 bp, Artificial Sequence
  Synthetic Primer
cagcattcaggaagaaagagg

SEQ ID NO: 12 - DNA, 20 bp, Artificial Sequence
  Synthetic Primer
gcttcttggatggaggtgac

SEQ ID NO: 13 - DNA, 24 bp, Artificial Sequence
  Synthetic Primer
aagccataacaacagtcttctgtg

SEQ ID NO: 14 - DNA, 20 bp, Artificial Sequence
  Synthetic Primer
gagatggccttgaagtcacc

SEQ ID NO: 15 - DNA, 20 bp, Artificial Sequence
  Synthetic Primer
ctgccaactcctttgtttgc

SEQ ID NO: 16 - DNA, 20 bp, Artificial Sequence
  Synthetic Primer
cggtttgcactccaatctct

SEQ ID NO: 17 - DNA, 20 bp, Artificial Sequence
  Synthetic Primer
aaggctcaaaccagacaagc

SEQ ID NO: 18 - DNA, 21 bp, Artificial Sequence
  Synthetic Primer
tcatgtgatgtccaggagttg